(19) United States
(12) Patent Application Publication
(10) Pub. No.: US 2011/0077272 A1
(43) Pub. Date: Mar. 31, 2011

(54) PULMONARY FORMULATIONS OF TRIPTANS

(75) Inventor: Mark Jonathan Main, Wiltshire (DE)

(73) Assignee: Vectura Limited, Wiltshire (GB)

(21) Appl. No.: 12/865,340

(22) PCT Filed: Feb. 2, 2009
(86) PCT No.: PCT/GB09/00265

§ 371 Date: Nov. 23, 2010

(30) Foreign Application Priority Data
Feb. 1, 2008 (GB) 0802024.0
Apr. 4, 2008 (GB) 0806156.6
Apr. 7, 2008 (GB) 0806283.8

(51) Int. Cl. A61K 31/454; C07D 209/16; A61K 31/404; C07D 403/10; A61K 31/4196; C07D 401/04; C07D 413/10; A61K 31/422; C07D 403/06; C07D 209/58; A61K 31/403; A61P 25/06

(52) U.S. Cl. 514/323; 548/504; 514/415; 548/266.4; 514/383; 546/201; 548/229; 514/376; 548/468; 514/414; 548/448; 514/411

(57) ABSTRACT

A pharmaceutical composition includes triptans, such as sumatriptan, and may be used in therapy. The composition may be administered via the inhaled route.

The present invention relates to pharmaceutical compositions comprising triptans, such as sumatriptan, and their uses in therapy. In particular, the invention relates to compositions for administration via the inhaled route.

### BACKGROUND TO THE INVENTION

Triptans are 5HT₁ receptor agonists which have been used in the treatment of migraine and special migraine type headaches like menstrual migraine, early morning migraine, as well as cluster headaches and tension type headaches. In addition they have been used to treat non-migraine headaches in migraineurs.

Triptans include sumatriptan, rizatriptan, naratriptan, zolmitriptan, eletriptan, almotriptan and frovatriptan.

The principle mode of action of triptans such as sumatriptan is thought to be the stimulation of the 5HT_{1B} receptor on the cranial vascular smooth muscle. This causes vasoconstriction which overcomes the pain induced by vasodilation which is thought to be responsible for headache. In addition, it is hypothesized that triptans stimulate a 5HT_{1D} receptor on pain fibres innervating the cranial vasculature which then blocks the release from the fibres of vasoactive peptides that cause neurogenic inflammation. It has also been shown that sumatriptan acts at the 5HT_{1F} receptor which may be important in mediating transmission of cranial pain information in the trigeminal nucleus caudalis. However, the clinical significance of sumatriptan's action at this receptor remains unknown at present (Dahlöf, C. G. H Curr Med Res Opin 17 (1s): s35-s45 2001).

Triptans are currently used for the acute treatment of migraine. Sumatriptan and zolmitriptan are currently marketed as orally administered products for treatment of migraine. Additionally, rizatriptan, sumatriptan and zolmitriptan have been formulated as fast melt formulations for rapid onset of action. Sumatriptan and zolmitriptan have also been formulated for nasal administration. Finally, sumatriptan has also been approved for subcutaneous administration.

The potential for severe adverse vascular events in patients with a higher risk for cardiovascular events precludes the unlimited use of triptans as a prophylactic treatment (Bigal et al., Medscape General Medicine, 2006, 8 (2) 31.). There is a very low, but definite potential risk of significant coronary vasoconstriction because of the presence of 5HT_{1B} receptors on peripheral and coronary vascular beds. Therefore, sumatriptan is contraindicated for patients with coronary heart disease. The side effects vary with the route of administration with the most intense effects being related to the subcutaneous injection (Sheftell et al., Expert Rev. Neurotherapeutics 4(2) 199-209 (2004)). The common side effects associated with the subcutaneous injection of sumatriptan are tingling, dizziness, drowsiness, transient increases in blood pressure soon after treatment, flushing, nausea and vomiting (but this may be due to the migraine), sensations of heaviness, mild injection site reactions, pain, sensations of heat, pressure or tightness which can effect any part of the body, usually transient but may be intense. Feelings of weakness and fatigue may also be experienced (GSK Imigran Injection SPC, 24 May 2006). Similar effects have been observed or 50 and 100 mg sumatriptan tablets. (GSK Imigran tablets 50 mg and 100 mg SPC, May 2006). Patients receiving the Imigran nasal spray have also reported the same side effects to the oral formulation in addition to bad taste and throat discomfort (GSK Imigran 10 mg and 20 mg Nasal Spray SPC 24 May 2006).

In addition to the foregoing issues associated with the known use of triptans, preclinical results have demonstrated that the pulmonary administration of sumatriptan may cause irritancy to the airways. Specifically, increased eosinophil counts may be considered indicative of irritant potential; at 1.9 mg/kg the respiratory changes were not of degenerative nature, whereas at 9 mg/kg degeneration occurred as observed from independent studies conducted by the applicant.

Furthermore, the disclosure in the past of vascular events, including deaths due to the vasoconstricting properties of triptans may discourage the skilled person from considering a pulmonary formulation. This is due to the potential for high, albeit transient, concentrations of sumatriptan in the pulmonary vein immediately after inhalation and the effect this might have on the coronary vasculature.

Early studies involving an intravenous route of administration were associated with a relatively high incidence of adverse effects, which were attributed to the rapid rise in plasma concentration after bolus infusion (Dechant et al, Drugs 43 (5) 776-798 1992). Other routes, such as subcutaneous delivery, were pursued as this allows for a comparatively slower, steadier delivery. Although a slower delivery method than intravenous, subcutaneous delivery still provides rapid relief beginning at 10 minutes after dosing. Other dosage forms are better tolerated, but are much slower. Oral tablets, for example, begin to alleviate headache after 30 minutes.

Research among migraine patients has shown the patients want complete relief of pain, no recurrence and rapid onset of action. Of these, speed of complete relief is the most important (Lipton R. B. et al headache 2002; 42 [suppl 1]: S3-9). The rapid onset of therapeutic effect is particularly beneficial for treating migraines as many sufferers experience an aura before the onset of the migraine itself. Prompt administration of a triptan with a rapid onset of action can, in some instances, avoid the onset of the actual migraine altogether. Whilst subcutaneous sumatriptan provides speed of relief, the disadvantages are the needle (for needle-phobic patients) and injection site reactions, which are the most common side effect. Injection site reactions occur in 59% of patients (Imitrex Prescribing Information November 2006).

Another disadvantage associated with the subcutaneous route of administration is that some patients suffer from impaired peripheral circulation during a migraine, which can reduce the efficacy of this delivery route (The Triptans: Novel Drugs for Migraine, edited by Humphrey P. et al Oxford University Press 2001).

Alternative routes of administration are better tolerated, but also have disadvantages. They are slower and less effective. Both oral and nasal forms suffer from erratic absorption. As migraine causes gastric stasis, this can adversely affect drug adsorption from an orally administered formulation such as a tablet. In addition nausea and vomiting make swallowing a tablet difficult. The nasal spray is often associated with a bad taste. Indeed, both nasal and oral administration of triptans can cause nausea and vomiting, particularly the latter, in a significant number of patients.

It is therefore an aim of the present invention to provide a pharmaceutical formulation comprising a triptan for administration in a manner that does not suffer from at least one and preferably more of the abovementioned disadvantages, whilst still providing a rapid onset of therapeutic action and relief, preferably with fewer adverse effects than are usually associated with the administration of triptans.

### SUMMARY OF THE INVENTION

According to a first aspect of the present invention, a pharmaceutical composition is provided comprising a triptan, for administration by pulmonary inhalation.

The present invention relates to high performance inhaled delivery of triptans, which has a number of significant and unexpected advantages over previously used routes of triptan administration. The route of administration and the compositions of the present invention make this excellent performance possible. The advantages of this pulmonary route of administration are improved safety, reduced exposure variability resulting in reduced incidence of adverse side effects, more rapid onset of action compared to subcutaneous and a non-invasive route of administration.

The triptans that are of particular interest include almotriptan, eletriptan, frovatriptan, naratriptan, rizatriptan, sumatriptan and zolmitriptan. Almotriptan is of particular interest as is it particularly well suited for use in inhalation on account of its lower incidence of side effects and lower activity on pulmonary arteries and veins. Sumatriptan is another preferred triptan.

In one embodiment of the present invention, the composition is a dry powder composition, preferably including active particles comprising the triptan.

Preferably, the composition is a dry powder which has a fine particle fraction (<5 μm) of at least 50%, preferably at least 60%, at least 70% or at least 80%.

In one embodiment of the present invention, the composition comprises active particles, at least 50%, at least 70%, at least 90% or substantially all of which have a Mass Median Aerodynamic Diameter (MMAD) of no more than about 10 μm. In another embodiment, at least 50%, at least 70%, at least 90% or substantially all of the active particles have an MMAD of from about 2 μm to about 5 μm. In yet another embodiment, at least 50%, at least 70% or at least 90% of the active particles have aerodynamic diameters in the range of about 0.05 μm to about 3 μm. In one embodiment of the invention, at least about 90% of the particles of the active agent, for example sumatriptan, have a particle size of 5 μm or less. Certain preferred compositions in accordance with the invention comprise active particles, at least 50%, at least 70%, at least 90% or substantially all of which have a diameter, preferably a MMAD, of at least 1, 1.1, 1.2, 1.5 or 2 μm.

In another embodiment, the powder compositions produced are within a size range greater than 10 μm and suitable for nasal delivery employing the technical disclosure discussed previously.

Whether intended for administration by inhalation or intranasally, the dry powder compositions of the present invention may benefit from including formulated particles of triptan (and any other pharmaceutically active material included) which are relatively dense particles. Thus, powders according to some embodiments of the present invention may preferably have a tapped density of more than 0.1 g/cc, more than 0.2 g/cc, more than 0.3 g/cc, more than 0.4 g/cc, more than 0.5 g/cc, more than 0.6 g/cc or more than 0.7 g/cc. The inclusion of such relatively dense particles of active material in dry powder compositions unexpectedly leads to good FPFs and FPDs and these dense particles may help reduce the volume of powder that must be administered to the lung or nasal mucosa. Especially in the case of intranasal administration, keeping the volume of powder to a minimum is beneficial, as it can help to reduce any discomfort felt by the patient. This is also of significant benefit where the dose of active agent to be administered is relatively large.

In an alternate embodiment, powders according to some embodiments of the present invention may preferably have a tapped density of more than 0.1 g/cc, more than 0.2 g/cc, more than 0.3 g/cc, more than 0.4 g/cc, more than 0.5 g/cc, more than 0.6 g/cc or more than 0.7 g/cc greater than the density of the active prior to processing.

The weight of the dry powder formulations according to the invention to be administered by inhalation may be as high as 20 mg (Delivered Dose).

According to a second aspect of the present invention, a pharmaceutical composition according to the first aspect of the present invention is provided, for the treatment or prophylaxis of conditions of the central nervous system, such as migraine.

The efficient and reproducible delivery of the active agent to the lung allows rapid absorption of an accurate and consistent amount to provide a predictable therapeutic effect. The efficient and reproducible delivery can be made more difficult where relatively large doses of the triptan must be administered and the ways in which the present invention overcomes these difficulties are set out in detail below.

In one embodiment of either of the first and second aspects of the invention, the triptan is sumatriptan. The sumatriptan used in these compositions can be in any suitable form, including salts of sumatriptan, most preferably sumatriptan succinate. The term “sumatriptan” as used herein includes the free base form of this compound as well as the pharmacologically acceptable salts or esters thereof. The free base of sumatriptan is particularly attractive in the context of the present invention as it crosses the lung barrier very readily and so it is anticipated that its administration via pulmonary inhalation will exhibit extremely fast onset of the therapeutic effect. Thus, any of the compositions disclosed herein may be formulated using the sumatriptan free base.

In addition to the succinate salt, other acceptable acid addition salts include the hydrobromide, the hydroiodide, the bisulfate, the phosphate, the acid phosphate, the lactate, the citrate, the tartrate, the salicylate, the maleate, the gluconate, and the like.

As used herein, the term “pharmaceutically acceptable esters” of sumatriptan refers to esters formed with one or both of the hydroxyl functions at positions 10 and 11, and which hydrolyse in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butryates, acrylates and ethyl succinates.

Typically, administration of a dose of the compositions according to the present invention will result in the delivery of a dose of about 3 to about 25 mg, and preferably of about 5 mg to about 20 mg of sumatriptan.

In another embodiment of the present invention, the dose of the powder composition delivers, in vitro, a fine particle dose of from about 0.4 mg to about 40 mg of sumatriptan (based on the weight of the succinate salt), when measured by a Multistage Liquid Impinger, United States Pharmacopoeia 26, Chapter 601, Apparatus 4 (2003), an Andersen Cascade Impactor or a New Generation Impactor.

In a preferred aspect, the present invention provides a pharmaceutical composition comprising a triptan, for administration by pulmonary inhalation, wherein said composition is to be administered in at least two sequential doses. Preferably, the sequential doses are administered within a period of no more than 5 minutes, 3 minutes, 2 minutes, 1 minute, or 30 seconds. It is preferred for the sequential doses to be of substantially the same size and, more preferably, for just two such doses to be administered. Preferably, said sequential doses are sufficient to provide a maximum serum concentration (Cₘₐₓ) of triptan that is in excess of double that provided by the administration of the first or a single such dose of the triptan when administered alone to the same subject. Preferably, each administered dose is of between 5 and 15 mg, 8 and 12 mg, 9 and 11 mg, 9.5 and 10.5 mg or about 10 mg of triptan. The triptan is preferably sumatriptan and, when it is, the latter doses are preferably based upon the weight of its succinate salt. More preferably, the triptan is sumatriptan succinate. Preferably the doses are metered doses (MD) or nominal doses (ND), alternatively they are delivered doses (DD) or emitted doses (ED). In an embodiment, the composition in accordance with this aspect of the invention is for the treatment or prophylaxis of conditions of the central nervous system, particularly migraine, special migraine type headaches like menstrual migraine and early morning migraine, cluster headaches or tension type headaches. In addition it can be for treating non-migraine headaches in migraineurs, but it is preferably used in the treatment of migraine. The composition in accordance with this aspect of the invention can be any pharmaceutical composition in accordance with the present invention, but it is preferably a dry powder composition.

In a related preferred aspect, the present invention provides method of treating a subject in need of therapy with a triptan, comprising administering to said subject a pharmaceutical composition comprising an effective amount of a triptan by pulmonary inhalation, wherein said composition is administered to said subject in at least two sequential doses. Preferably, the sequential doses are administered within a period of no more than 5 minutes, 3 minutes, 2 minutes, 1 minute, or 30 seconds. It is preferred for the sequential doses to be of substantially the same size and, more preferably, for just two such doses to be administered. Preferably, said sequential doses are sufficient to provide a maximum serum concentration (Cₘₐₓ) of triptan that is in excess of double that provided by the administration of the first or a single such dose of the triptan when administered alone to the same subject. Preferably, each administered dose is of between 5 and 15 mg, 8 and 12 mg, 9 and 11 mg, 9.5 and 10.5 mg or about 10 mg of triptan. The triptan is preferably sumatriptan and, when it is, the latter doses are preferably based upon the weight of its succinate salt. More preferably, the triptan is sumatriptan succinate. Preferably the doses are metered doses (MD) or nominal doses (ND), alternatively they are delivered doses (DD) or emitted doses (ED). In an embodiment, the method in accordance with this aspect of the invention is for the treatment or prophylaxis of conditions of the central nervous system, particularly migraine, special migraine type headaches like menstrual migraine and early morning migraine, cluster headaches or tension type headaches. In addition it can be for treating non-migraine headaches in migraineurs, but it is preferably used in the treatment of migraine. The composition used in accordance with this aspect of the invention can be any pharmaceutical composition in accordance with the present invention, but it is preferably a dry powder composition.

An unexpected advantage of the last two described embodiments of the invention is that they provide much greater peak serum drug concentrations (Cₘₐₓ), and drug bioavailability (AUC), than would be expected from an equivalent single dose, whilst not inducing any significant levels of adverse side effects. Indeed, the last two described embodiments allow incidents of nausea and vomiting to be reduced (this also applies to all inhaled compositions in accordance with the invention).

In a preferred embodiment, at least some of the triptan is in amorphous form. A formulation containing amorphous triptan will possess preferable dissolution characteristics. A stable form of amorphous triptan may be prepared using suitable sugars such as trehalose and melezitose by spray drying as exemplified below. Preferably the amorphous triptan is amorphous sumatriptan.

In a preferred embodiment, the formulation or pharmaceutical composition may comprise two or more triptans. For example, sumatriptan may be combined with slower acting triptans like frovatriptan or naratriptan to provide a combination which has the benefit of rapid onset of action (afforded by the sumatriptan) but also conveying the benefit of low recurrence due to their longer half life (afforded by the frovatriptan or naratriptan).

In some embodiments, the compositions of the present invention comprise active particles, preferably comprising sumatriptan, and carrier particles. The carrier particles may have an average particle size of from about 5 to about 1000 μm, from about 4 to about 40 μm, from about 60 to about 200 μm, or from 150 to about 1000 μm. Other useful average particle sizes for carrier particles are about 20 to about 30 μm or from about 40 to about 70 μm.

Preferably, the carrier particles are present in small amount, such as no more than 90%, preferably 80%, more preferably 70%, more preferably 60% more preferably 50% by weight of the total composition. In such “low carrier” compositions, the composition preferably also includes at least small amounts of additive materials, to improve the powder properties and performance.

In certain embodiments of the present invention, the compositions are “carrier free”, which means that they include substantially only the triptan, such as sumatriptan or one of its pharmaceutically acceptable salts or esters, and one or more additive materials.

In a further embodiment, the composition comprises at least about 70% (by weight) triptan, or at least about 75%, 80%, 85%, 90%, 95%, 96%, 97%, 98% or 99% (by weight) triptan.

The compositions according to the invention may further include one or more additive materials. The additive material may be in the form of particles which tend to adhere to the surfaces of the active particles, as disclosed in WO 97/03649. Alternatively, the additive material may be coated on the surface of the active particles by, for example a co-milling method as disclosed in WO 02/43701 or on the surfaces of the carrier particles, as disclosed in WO 02/00197.

Alternatively or in addition, the additive material may be coated onto the surface of carrier particles present in the composition. This additive coating may be in the form of particles of additive material adhering to the surfaces of the carrier particles (by virtue of interparticle forces such as Van der Waals forces), as a result of a blending of the carrier and additive. Alternatively, the additive material may be smeared over and fused to the surfaces of the carrier particles, thereby forming composite particles with a core of inert carrier material and additive material on the surface. For example, such fusion of the additive material to the carrier particles may be achieved by co-jet milling particles of additive material and carrier particles. In some embodiments, all three components of the powder (active, carrier and additive) are processed together so that the additive becomes attached to or fused to both the carrier particles and the active particles.

According to a third aspect of the present invention, blisters, capsules, reservoir dispensing systems and the like are provided, comprising doses of the compositions according to the invention.

According to a fourth aspect of the present invention, inhaler devices are provided for dispensing doses of the compositions according to the invention. In one embodiment of the present invention, the inhalable compositions are administered via a dry powder inhaler (DPI). In an alternative embodiment, the compositions are administered via a pressurized metered dose inhaler (pMDI), or via a nebulised system.

According to a fifth aspect of the present invention, processes are provided for preparing the compositions according to the invention.

In one embodiment, the compositions according to the present invention are prepared by simply blending particles of triptan of a selected appropriate size with particles of other powder components, such as additive and/or carrier particles. The powder components may be blended by a gentle mixing process, for example in a tumble mixer such as a Turbula®. In such a gentle mixing process, there is generally substantially no reduction in the size of the particles being mixed. In addition, the powder particles do not tend to become fused to one another, but they rather agglomerate as a result of cohesive forces such as Van der Waals forces. These loose or unstable agglomerates readily break up upon actuation of the inhaler device used to dispense the composition.

In one embodiment, if required, the microparticles produced by the milling step can then be formulated with an additional excipient. This may be achieved by a spray drying process, e.g. co-spray drying with excipients. In this embodiment, the particles are suspended in a solvent and co-spray dried with a solution or suspension of the additional excipient. Preferred additional excipients include trehalose, melezitose and other polysaccharides. Additional pharmaceutical effective excipients may also be used.

In another embodiment, the powder compositions are produced using a multi-step process. Firstly, the materials are milled or blended. Next, the particles may be sieved, prior to undergoing a controlled compressive milling step such as mechanofusion or mechano-chemical bonding. A further optional step involves the addition of carrier particles. The mechanofusion step is thought to “polish” the composite active particles, further rubbing the additive material into the active particles. This allows one to enjoy the beneficial properties afforded to particles by a controlled compressive milling step such as mechanofusion or mechano-chemical bonding, in combination with the very small particles sizes made possible by the jet milling.

According to a sixth aspect of the present invention, methods for the treatment or prophylaxis of conditions of the central nervous system, such as migraine, are provided, the methods involving administering doses of the compositions according to the invention by pulmonary inhalation.

### DETAILED DESCRIPTION OF THE INVENTION

Preferably, for delivery to the lower respiratory tract or deep lung, the mass median aerodynamic diameter (MMAD) of the active particles in a dry powder composition is not more than 10 μm, and preferably not more than 5 μm, more preferably not more than 3 μm, and may be less than 2 μm, less than 1.5 μm or less than 1 μm. Especially for deep lung or systemic delivery, the active particles may have a size of 0.1 to 3 μm or 0.1 to 2 μm.

Ideally, at least 90% by weight of the active particles in a dry powder formulation should have an aerodynamic diameter of not more than 10 μm, preferably not more than 5 μm, more preferably not more than 3 μm, not more than 2.5 μm, not more than 2.0 μm, not more than 1.5 μm, or even not more than 1.0 μm.

The particles of active agent included in the compositions of the present invention, may be formulated with additional excipients to aid delivery or to control release of the active agent upon deposition within the lung. In such embodiments, the active agent may be embedded in or dispersed throughout particles of an excipient material which may be, for example, a polysaccharide matrix. Alternatively, the excipient may form a coating, partially or completely surrounding the particles of active material. Upon delivery of these particles to the lung, the excipient material acts as a temporary barrier to the release of the active agent, providing a delayed or sustained release of the active agent. Suitable excipient materials for use in delaying or controlling the release of the active material will be well known to the skilled person and will include, for example, pharmaceutically acceptable soluble or insoluble materials such as polysaccharides, for example xanthan gum. A dry powder composition may comprise the active agent in the form of particles which provide immediate release, as well as particles exhibiting delayed or sustained release, to provide any desired release profile.

When dry powders are produced using conventional processes, the active particles will vary in size, and often this variation can be considerable. This can make it difficult to ensure that a high enough proportion of the active particles are of the appropriate size for administration to the correct site. In certain circumstances it may therefore be desirable to have a dry powder formulation wherein the size distribution of the active particles is narrow. For example, the geometric standard deviation of the active particle aerodynamic or volumetric size distribution (ag), may preferably be not more than 2, more preferably not more than 1.8, not more than 1.6, not more than 1.5, not more than 1.4, or even not more than 1.2. A narrow particle size distribution may be of particular importance in view of sumatriptan's narrow therapeutic index. A narrow particle size ensures that doses are both reproducible with respect to sumatriptan content and that the dose is delivered to the same region of the lung on each delivery ensuring a reproducible pharmacokinetic profile. This may improve dose efficiency and reproducibility.

Fine particles, that is, those with a Mass Median Aerodynamic Diameter (MMAD) of less than 10 μm, tend to be increasingly thermodynamically unstable as their surface area to volume ratio increases, which provides an increasing surface free energy with this decreasing particle size, and consequently increases the tendency of particles to agglomerate and the strength of the agglomerate. In the inhaler, agglomeration of fine particles and adherence of such particles to the walls of the inhaler are problems that result in the fine particles leaving the inhaler as large, stable agglomerates, or being unable to leave the inhaler and remaining adhered to the interior of the inhaler, or even clogging or blocking the inhaler.

The uncertainty as to the extent of formation of stable agglomerates of the particles between each actuation of the inhaler, and also between different inhalers and different batches of particles, leads to poor dose reproducibility. Furthermore, the formation of agglomerates means that the MMAD of the active particles can be vastly increased, with agglomerates of the active particles not reaching the required part of the lung.

In an attempt to improve this situation and to provide a consistent Fine Particle Fraction (FPF) and Fine Particle Dose (FPD), dry powder formulations often include additive material. The additive material is intended to control the cohesion between particles in the dry powder formulation. It is thought that the additive material interferes with the weak bonding forces between the small particles, helping to keep the particles separated and reducing the adhesion of such particles to one another, to other particles in the formulation if present and to the internal surfaces of the inhaler device. Where agglomerates of particles are formed, the addition of particles of additive material decreases the stability of those agglomerates so that they are more likely to break up in the turbulent air stream created on actuation of the inhaler device, whereupon the particles are expelled from the device and inhaled. As the agglomerates break up, the active particles return to the form of small individual particles which are capable of reaching the lower lung.

However, the optimum stability of agglomerates to provide efficient drug delivery will depend upon the nature of the turbulence created by the particular device used to deliver the powder. Agglomerates will need to be stable enough for the powder to exhibit good flow characteristics during processing and loading into the device, whilst being unstable enough to release the active particles of respirable size upon actuation.

In the past, many of the commercially available dry powder inhalers exhibited very poor dosing efficiency, with sometimes as little as 10% of the active agent present in the dose actually being properly delivered to the user so that it can have a therapeutic effect. This low efficiency is simply not acceptable where a high dose of active agent is required for the desired therapeutic effect.

The reason for the lack of dosing efficiency is that a proportion of the active agent in the dose of dry powder tends to be effectively lost at every stage the powder goes through from expulsion from the delivery device to deposition in the lung. For example, substantial amounts of material may remain in the blister/capsule or device. Material may be lost in the throat of the subject due to excessive plume velocity. However, it is frequently the case that a high percentage of the dose delivered exists in particulate forms of aerodynamic diameter in excess of that required.

It is well known that particle impaction in the upper airways of a subject is predicted by the so-called impaction parameter. The impaction parameter is defined as the velocity of the particle multiplied by the square of its aerodynamic diameter. Consequently, the probability associated with delivery of a particle through the upper airways region to the target site of action, is related to the square of its aerodynamic diameter. Therefore, delivery to the lower airways, or the deep lung is dependent on the square of its aerodynamic diameter, and smaller aerosol particles are very much more likely to reach the target site of administration in the user and therefore able to have the desired therapeutic effect.

Particles having aerodynamic diameters of less than 10 μm tend to be deposited in the lung. Particles with an aerodynamic diameter in the range of 2 μm to 5 μm will generally be deposited in the respiratory bronchioles whereas smaller particles having aerodynamic diameters in the range of 0.05 to 3 μm are likely to be deposited in the alveoli. So, for example, high dose efficiency for particles targeted at the alveoli is predicted by the dose of particles below 3 μm, with the smaller particles being most likely to reach that target site.

The metered dose (MD), also known as the Nominal Dose (ND), of a dry powder composition is the total mass of active agent present in the metered form presented by the inhaler device in question i.e. the amount of drug metered in the dosing receptacle or container. For example, the MD might be the mass of active agent present in a capsule for a Cyclohaler™, or in a foil blister in a Gyrohaler™ device or powder indentations of a ClickHaler™. The MD is different to the amount of drug that is delivered to the patient which is referred to a Delivered Dose (DD) or Emitted Dose (ED). These terms are used interchangeably herein and they are measured as set out in the current EP monograph for inhalation products.

The emitted dose (ED) is the total mass of the active agent emitted from the device following actuation. It does not include the material left on the internal or external surfaces of the device, or in the metering system including, for example, the capsule or blister. The ED is measured by collecting the total emitted mass from the device in an apparatus frequently identified as a dose uniformity sampling apparatus (DUSA), and recovering this by a validated quantitative wet chemical assay (a gravimetric method is possible, but this is less precise).

The fine particle dose (FPD) is the total mass of active agent which is emitted from the device following actuation which is present in an aerodynamic particle size smaller than a defined limit. This limit is generally taken to be 5 μm if not expressly stated to be an alternative limit, such as 3 μm, 2 μm or 1 μm, etc. The FPD is measured using an impactor or impinger, such as a twin stage impinger (TSI), multi-stage impinger (MSI), Andersen Cascade Impactor (ACI) or a Next Generation Impactor (NGI). Each impactor or impinger has a pre-determined aerodynamic particle size collection cut points for each stage. The FPD value is obtained by interpretation of the stage-by-stage active agent recovery quantified by a validated quantitative wet chemical assay (a gravimetric method is possible, but this is less precise) where either a simple stage cut is used to determine FPD or a more complex mathematical interpolation of the stage-by-stage deposition is used.

The fine particle fraction (FPF) is normally defined as the FPD (the dose that is <5 μm) divided by the Emitted Dose (ED) which is the dose that leaves the device. The FPF is expressed as a percentage. Herein, the FPF of ED is referred to as FPF (ED) and is calculated as FPF (ED)=(FPD/ED)×100%.

The fine particle fraction (FPF) may also be defined as the FPD divided by the Metered Dose (MD) which is the dose in the blister or capsule, and expressed as a percentage. Herein, the FPF of MD is referred to as FPF (MD), and may be calculated as FPF (MD)=(FPD/MD)×100%.

The term “ultrafine particle dose” (UFPD) is used herein to mean the total mass of active material delivered by a device which has a diameter of not more than 3 μm. The term “ultrafine particle fraction” is used herein to mean the percentage of the total amount of active material delivered by a device which has a diameter of not more than 3 μm. The term percent ultrafine particle dose (% UFPD) is used herein to mean the percentage of the total metered dose which is delivered with a diameter of not more than 3 μm (i.e., % UFPD=100×UFPD/total metered dose).

The uncertainty as to the extent of formation of stable agglomerates of the particles between each actuation of the inhaler, and also between different inhalers and different batches of particles, leads to poor dose reproducibility. Furthermore, the formation of agglomerates means that the MMAD of the active particles can be vastly increased, with agglomerates of the active particles not reaching the required part of the lung. Consequently, it is essential for the present invention to provide a powder formulation which provides good dosing efficiency and reproducibility, delivering an accurate and predictable dose.

Much work has been done to improve the dosing efficiency of dry powder systems comprising active particles having a size of less than 10 μm, reducing the loss of the pharmaceutically active agent at each stage of the delivery. In the past, efforts to increase dosing efficiency and to obtain greater dosing reproducibility have tended to focus on preventing the formation of agglomerates of fine particles of active agent. Such agglomerates increase the effective size of these particles and therefore prevent them from reaching the lower respiratory tract or deep lung, where the active particles should be deposited in order to have their desired therapeutic effect. Proposed measures have included the use of relatively large carrier particles. The fine particles of active agent tend to become attached to the surfaces of the carrier particles as a result of interparticle forces such as Van der Waals forces. Upon actuation of the inhaler device, the active particles are supposed to detach from the carrier particles and are then present in the aerosol cloud in inhalable form. In addition or as an alternative, the inclusion of additive materials that act as force control agents that modify the cohesion and adhesion between particles has been proposed.

However, where the dose of drug to be delivered is very high, the options for adding materials to the powder composition are limited. This is especially true where at least 70% of the compositions has comprise the active agent, as is the case with some of the preferred triptans used in the present invention. Nevertheless, it is imperative that the dry powder composition exhibit good flow and dispersion properties, to ensure good dosing efficiency.

A number of measures may be taken to ensure that the compositions according to the present invention have good flow and dispersion properties and these are discussed below. One or more of these measures may be adopted in order to obtain a composition with properties that ensure efficient and reproducible drug delivery to the lung.

Powder Components

Force Control Agents

The compositions according to the present invention may include additive materials that control the cohesion and adhesion of the particles of the powder.

The tendency of fine particles to agglomerate means that the FPF of a given dose can be highly unpredictable and a variable proportion of the fine particles will be administered to the lung, or to the correct part of the lung, as a result. This is observed, for example, in formulations comprising pure drug in fine particle form. Such formulations exhibit poor flow properties and poor FPF.

In an attempt to improve this situation and to provide a good consistent FPF and FPD, dry powder compositions according to the present invention may include additive material which is an anti-adherent material and whose presence on the surface of a particle can modify the adhesive and cohesive surface forces experienced by that particle, in the presence of other particles and in relation to the surfaces that the particles are exposed to. In general, its function is to reduce both the adhesive and cohesive forces. These additives are therefore sometimes referred to as force control agents (FCAs).

It is thought that the FCAs interfere with the weak bonding forces between the small particles, helping to keep the particles separated and reducing the adhesion of such particles to one another, to other particles in the formulation if present and to the internal surfaces of the inhaler device. Where agglomerates of particles are formed, the addition of particles of FCA decreases the stability of those agglomerates so that they are more likely to break up in the turbulent air stream created on actuation of the inhaler device, whereupon the particles are expelled from the device and inhaled. As the agglomerates break up, the active particles may return to the form of small individual particles or agglomerates of small numbers of particles which are capable of reaching the lower lung.

The additive material or FCA may be in the form of particles which tend to adhere to the surfaces of the active particles, as disclosed in WO 97/03649. Alternatively, it may be coated on the surface of the active particles by, for example a co-milling method as disclosed in WO 02/43701.

Advantageously, the FCA is an anti-friction agent or glidant and will give the powder formulation better flow properties in the inhaler. The materials used in this way may not necessarily be usually referred to as anti-adherents or anti-friction agents, but they will have the effect of decreasing the cohesion between the particles or improving the flow of the powder and they usually lead to better dose reproducibility and higher FPFs.

The reduced tendency of the particles to bond strongly, either to each other or to the device itself, not only reduces powder cohesion and adhesion, but can also promote better flow characteristics. This leads to improvements in the dose reproducibility because it reduces the variation in the amount of powder metered out for each dose and improves the release of the powder from the device. It also increases the likelihood that the active material, which does leave the device, will reach the lower lung of the patient.

It is favourable for unstable agglomerates of particles to be present in the powder when it is in the inhaler device. For a powder to leave an inhaler device efficiently and reproducibly, the particles of such a powder should be large, preferably larger than about 40 μm. Such a powder may be in the form of either individual particles having a size of about 40 μm or larger and/or agglomerates of finer particles, the agglomerates having a size of about 40 μm or larger. The agglomerates formed can have a size of 100 μm or 200 μm and, depending on the type of device used to dispense the formulation, the agglomerates may be as much as about 1000 μm. With the addition of the FCA, those agglomerates are more likely to be broken down efficiently in the turbulent airstream created on inhalation. Therefore, the formation of unstable or “soft” agglomerates of particles in the powder may be favoured compared with a powder in which there is substantially no agglomeration. Such unstable agglomerates are stable whilst the powder is inside the device but are then disrupted and broken up upon inhalation.

It is particularly advantageous for the FCA to comprise, for example, metal stearates such as magnesium stearate, phospholipids, lecithin, colloidal silicon dioxide and sodium stearyl fumarate, and are described more fully in WO 96/23485, which is hereby incorporated by reference.

Advantageously, the powder includes at least 80%, preferably at least 90% and most preferably at least 95% by weight of triptan (or its pharmaceutically acceptable salts) based on the weight of the powder. The optimum amount of additive material or FCA will depend upon the precise nature of the material used and the manner in which it is incorporated into the composition. In some embodiments, the powder advantageously includes not more than 8%, more advantageously not more than 5%, more advantageously not more than 3%, more advantageously not more than 2%, more advantageously not more than 1%, and more advantageously not more than 0.5% by weight of FCA based on the weight of the powder. As indicated above, in some cases it will be advantageous for the powder to contain about 1% by weight of FCA. In other embodiments, the FCA may be provided in an amount from about 0.1% to about 10% by weight, and preferably from about 0.5% to 8%, most preferably from about 1% to about 5%.

When the FCA is micronised leucine or lecithin, it is preferably provided in an amount from about 0.1% to about 10% by weight. Preferably, the FCA comprises from about 3% to about 7%, preferably about 5%, of micronised leucine. Preferably, at least 95% by weight of the micronised leucine has a particle diameter of less than 150 μm, preferably less than 100 μm, and most preferably less than 50 μm. Preferably, the mass median diameter of the micronised leucine is less than 10 μm.

If magnesium stearate or sodium stearyl fumarate is used as the FCA, it is preferably provided in an amount from about 0.05% to about 10%, from about 0.15% to about 7%, from about 0.25% to about 6%, or from about 0.5% to about 5% depending on the required final dose.

Known FCAs usually consist of physiologically acceptable material, although the additive material may not always reach the lung. Preferred FCAs for used in dry powder compositions include amino acids, peptides and polypeptides having a molecular weight of between 0.25 and 1000 kDa and derivatives thereof.

The FCA may comprise or consist of dipolar ions, which may be zwitterions. It is also advantageous for the FCA to comprise or consist of a spreading agent, to assist with the dispersal of the composition in the lungs. Suitable spreading agents include surfactants such as known lung surfactants (e.g. ALEC®) which comprise phospholipids, for example, mixtures of DPPC (dipalmitoyl phosphatidylcholine) and PG (phosphatidylglycerol). Other suitable surfactants include, for example, dipalmitoyl phosphatidylethanolamine (DPPE), dipalmitoyl phosphatidylinositol (DPPI).

It is particularly advantageous for the FCA to comprise of a metal stearate, for example, zinc stearate, magnesium stearate, calcium stearate, sodium stearate or lithium stearate, or a derivative thereof, for example, sodium stearyl fumarate or sodium stearyl lactylate. It is particularly advantageous for the FCA to exhibit glidant properties to the pharmaceutical composition.

The FCA may comprise or consist of one or more surface active materials, in particular materials that are surface active in the solid state, which may be water soluble or water dispersible, for example lecithin, in particular soya lecithin, or substantially water insoluble, for example solid state fatty acids such as oleic acid, lauric acid, palmitic acid, stearic acid, erucic acid, behenic acid, or derivatives (such as esters and salts) thereof, such as glyceryl behenate. Specific examples of such surface active materials are phosphatidylcholines, phosphatidylethanolamines, phosphatidylglycerols and other examples of natural and synthetic lung surfactants; lauric acid and its salts, for example, sodium lauryl sulphate, magnesium lauryl sulphate; triglycerides such as Dynsan 118 and Cutina HR; and sugar esters in general. Alternatively, the FCA may comprise or consist of cholesterol. Other useful FCAs are film-forming agents, fatty acids and their derivatives, as well as lipids and lipid-like materials. In some embodiments, a plurality of different FCAs can be used.

In one preferred embodiment, the composition includes an FCA, such as magnesium stearate (up to 10% w/w) or leucine, said FCA being jet-milled with the particles of triptan, preferably with particles of sumatriptan.

Advantageously, in the “carrier free” formulations, at least 90% by weight of the particles of the powder have a particle size less than 63 μm, preferably less than 30 μm and more preferably less than 10 μm. As indicated above, the size of the particles of triptan (or its pharmaceutically acceptable salts) in the powder should be within the range of about from 0.1 μm to 5 μm for effective delivery to the lower lung. Where the additive material is in particulate form, it may be advantageous for these additive particles to have a size outside the preferred range for delivery to the lower lung.

In some embodiments, the powder composition includes at least 60% by weight of the triptan or a pharmaceutically acceptable salt or ester thereof based on the weight of the powder. Advantageously, the powder comprises at least 70%, or at least 80% by weight of triptan or a pharmaceutically acceptable salt or ester thereof based on the weight of the powder. Most advantageously, the powder comprises at least 90%, at least 93%, or at least 95% by weight of triptan or a pharmaceutically acceptable salt or ester thereof based on the weight of the powder. It is believed that there are physiological benefits in introducing as little powder as possible to the lungs, in particular material other than the active ingredient to be administered to the patient. Therefore, the quantities in which the additive material is added are preferably as small as possible. The most preferred powder, therefore, would comprise more than 95% by weight of triptan or a pharmaceutically acceptable salt or ester thereof. Preferably, the triptan is sumatriptan.

In a specific embodiment, the formulation does not contain carrier particles and comprises triptan and an FCA, such as at least 30%, preferably 60%, more preferably 80%, more preferably 90% more preferably 95% and most preferably 97% by weight of the total composition comprises the pharmaceutically active agent. The active agent may be a triptan alone, such as sumatriptan, or it may be a combination of a triptan with secondary active wherein said active is used to reduced any adverse and unwanted secondary effects which would benefit migraine patients. The remaining components may comprise one or more additive materials, such as those discussed above.

Carrier Particles

Another approach for improving the flow and dispersion properties of the compositions of the present invention is the inclusion of carrier particles.

In a further attempt to improve extraction of the dry powder from the dispensing device and to provide a consistent FPF and FPD, dry powder compositions according to the present invention may include carrier particles of an inert excipient material, mixed with fine particles of active material. In such compositions, especially where the carrier particles are larger than the active particles, rather than sticking to one another, the fine active particles tend to adhere to the surfaces of the carrier particles whilst in the inhaler device, but are supposed to release and become dispersed upon actuation of the dispensing device and inhalation into the respiratory tract, to give a fine suspension. Such release may be improved by the inclusion of an additive material, such as an FCA as discussed above.

The inclusion of carrier particles is less attractive where very large doses of active agent are to be delivered, as they tend to significantly increase the volume of the powder composition. Nevertheless, in some embodiments of the present invention, the compositions include carrier particles.

Carrier particles may comprise or consist of any acceptable excipient material or combination of materials and preferably the material(s) is (are) inert and physiologically acceptable. For example, the carrier particles may be composed of one or more materials selected from sugar alcohols, polyols and crystalline sugars. Other suitable carriers include inorganic salts such as sodium chloride and calcium carbonate, organic salts such as sodium lactate and other organic compounds such as polysaccharides and oligosaccharides. Advantageously the carrier particles are of a polyol. In particular the carrier particles may be particles of crystalline sugar, for example mannitol, trehalose, melizitose, dextrose or lactose. Preferably, the carrier particles comprise or consist of lactose.

According to some embodiments of the present invention, the dry powder compositions include carrier particles that are relatively large, compared to the particles of active material. This means that substantially all (by weight) of the carrier particles have a diameter which lies between 20 μm and 1000 μm, or between 50 μm and 1000 μm. Preferably, the diameter of substantially all (by weight) of the carrier particles is less than 355 μm and lies between 20 μm and 250 μm. In one embodiment, the carrier particles have a MMAD of at least 90 μm.

Preferably, at least 90% by weight of the carrier particles have a diameter between from 60 μm to 180 μm. The relatively large diameter of the carrier particles improves the opportunity for other, smaller particles to become attached to the surfaces of the carrier particles and to provide good flow and entrainment characteristics and improved release of the active particles in the airways to increase deposition of the active particles in the lower lung.

In another embodiment of the present invention, the carrier particles may have an average particle size of from about 5 to about 1000 μm, from about 4 to about 40 μm, from about 60 to about 200 μm, or from 150 to about 1000 μm. Other useful average particle sizes for carrier particles are about 20 to about 30 μm or from about 40 to about 70 μm.

Powder flow problems associated with compositions comprising larger amounts of fine material, such as up to from 5 to 20% by total weight of the formulation. This problem may be overcome by the use of large fissured lactose carrier particles, as discussed in earlier patent applications published as WO 01/78694, WO 01/78695 and WO 01/78696.

In other embodiments, the excipient or carrier particles included in the dry powder compositions are relatively small, having a median diameter of about 3 to about 40 μm, preferably about 5 to about 30 μm, more preferably about 5 to about 20 μm, and most preferably about 5 to about 15 μm. Such fine carrier particles, if untreated with an additive are unable to provide suitable flow properties when incorporated in a powder composition comprising fine or ultra-fine active particles. Indeed, previously, particles in these size ranges would not have been regarded as suitable for use as carrier particles, and instead would only have been added in small quantities as a fine component in combination with coarse carrier particles, in order to increase the aerosolisation properties of compositions containing a drug and a larger carrier, typically with median diameter 40 μm to 100 μm or greater. However, the quantity of such a fine excipient may be increased and such fine excipient particles may act as carrier particles if these particles are treated with an additive or FCA, even in the absence of coarse carrier particles. Such treatment can bring about substantial changes in the powder characteristics of the fine excipient particles and the powders they are included in. Powder density is increased, even doubled, for example from 0.3 g/cm³ to over 0.5 g/cm³. Other powder characteristics are changed, for example, the angle of repose is reduced and contact angle increased.

Treated fine carrier particles having a median diameter of 3 to 40 μm are advantageous as their relatively small size means that they have a reduced tendency to segregate from the drug component, even when they have been treated with an additive to reduce cohesion. This is because the size differential between the carrier and drug is relatively small compared to that in conventional compositions which include fine or ultra-fine active particles and much larger carrier particles. The surface area to volume ratio presented by the fine carrier particles is correspondingly greater than that of conventional large carrier particles. This higher surface area, allows the carrier to be successfully associated with higher levels of drug than for conventional larger carrier particles. This makes the use of treated fine carrier particles particularly attractive in powder compositions to be dispensed by passive devices.

Carrier-based systems can be particularly advantageous when formulating with uncoated particles of active agent as described above. Such “uncoated systems” are particularly desirable when rapid onset of action is required. Uncoated systems are possible without carriers, however, for reasons outlined above, the feasibility largely depends on the precise chemical and physical makeup of the active. Depending on the precise nature of a coated system, it is possible to delay the dissolution of the drug for example, the more additive used the less the rate of drug dissolution.

Preferably, the carrier particles are present in small amount, such as no more than 90%, preferably 80%, more preferably 70%, more preferably 60% more preferably 50% by weight of the total composition.

In one embodiment, the composition comprises approximately 50% carrier particles, 45% triptan and 5% FCA. In an alternate specific embodiment, the composition comprises approximately 80% carrier particles 18% triptan and 2% FCA. As the amount of carrier in these formulations changes, the amounts of additive and triptan will also change, but the ratio of these constituents preferably remains approximately 1:9. Preferably, the triptan is sumatriptan and the FCA is magnesium stearate.

In a further embodiment, the formulation comprises at least 30%, at least 60%, at least 80%, at least 90%, at least 95% or at least 97% by weight of the total composition comprises the pharmaceutically active agent and wherein the remaining components comprise additive material and carrier particles. The larger particles provide the dual action of acting as a carrier and facilitating powder flow.

In a specific embodiment, the composition comprises triptan (30% w/w) and lactose having an average particles size of 45-65 μm. The compositions comprising triptan and carrier particles may further include one or more additive materials. The additive material, which may be an FCA as discussed above, may be in the form of particles which tend to adhere to the surfaces of the active particles, as disclosed in WO 97/03649. Alternatively, the additive material may be coated on the surface of the active particles by, for example a co-milling method as disclosed in WO 02/43701 or on the surfaces of the carrier particles, as disclosed in WO 02/00197.

Powder Preparation

Other measures that may be taken to ensure that the compositions according to the present invention have good flow and dispersion properties involve the preparation or processing of the powder particles, and in particular of the active particles which comprise the triptan. Examples of appropriate formulation approaches are set out in more detail below.

Spray Drying

Spray drying may be used to produce particles of inhalable size comprising the triptan. The spray drying process may be adapted to produce spray-dried particles that include the active agent and an additive material which controls the agglomeration of particles and powder performance. The spray drying process may also be adapted to produce spray-dried particles that include the active agent dispersed or suspended within a material that provides the controlled release properties.

Conventional spray drying of triptan often results in a triptan “jelly”. These conventional spray drying techniques may be improved so as to produce active particles with enhanced chemical and physical properties so that they perform better when dispensed from a DPI than particles formed using conventional spray drying techniques. Some of such improvements are described in detail in the earlier patent application published as WO 2005/025535.

Furthermore the dispersal or suspension of the active material within an excipient material may impart further stability to the active compounds. In a preferred embodiment the triptan, such as sumatriptan, may reside primarily in the amorphous state. A formulation containing amorphous triptan will possess preferable dissolution characteristics. This would be possible in that particles are suspended in a sugar glass which could be either a solid solution or a solid dispersion. Preferred additional excipients include trehalose, melezitose and other polysaccharides.

In particular, it is disclosed that co-spray drying an active agent with an FCA under specific conditions can result in particles with excellent properties which perform extremely well when administered by a DPI for inhalation into the lung.

It has been found that manipulating or adjusting the spray drying process can result in the FCA being largely present on the surface of the particles. That is, the FCA is concentrated at the surface of the particles, rather than being homogeneously distributed throughout the particles. This clearly means that the FCA will be able to reduce the tendency of the particles to agglomerate. This will assist the formation of unstable agglomerates that are easily and consistently broken up upon actuation of a DPI.

It has been found that it may be advantageous to control the formation of the droplets in the spray drying process, so that droplets of a given size and of a narrow size distribution are formed. Furthermore, controlling the formation of the droplets can allow control of the air flow around the droplets which, in turn, can be used to control the drying of the droplets and, in particular, the rate of drying. Controlling the formation of the droplets may be achieved by using alternatives to the conventional 2-fluid nozzles, especially avoiding the use of high velocity air flows.

In particular, it is preferred to use a spray drier comprising a means for producing droplets moving at a controlled velocity and of a predetermined droplet size. The velocity of the droplets is preferably controlled relative to the body of gas into which they are sprayed. This can be achieved by controlling the droplets' initial velocity and/or the velocity of the body of gas into which they are sprayed, for example by using an ultrasonic nebuliser (USN) to produce the droplets. Alternative nozzles such as electrospray nozzles or vibrating orifice nozzles may be used.

In one embodiment, an ultrasonic nebuliser (USN) is used to form the droplets in the spray mist. USNs use an ultrasonic transducer which is submerged in a liquid. The ultrasonic transducer (a piezoelectric crystal) vibrates at ultrasonic frequencies to produce the short wavelengths required for liquid atomisation. In one common form of USN, the base of the crystal is held such that the vibrations are transmitted from its surface to the nebuliser liquid, either directly or via a coupling liquid, which is usually water. When the ultrasonic vibrations are sufficiently intense, a fountain of liquid is formed at the surface of the liquid in the nebuliser chamber. Droplets are emitted from the apex and a “fog” emitted.

Whilst ultrasonic nebulisers are known, these are conventionally used in inhaler devices, for the direct inhalation of solutions containing drug, and they have not previously been widely used in a spray drying apparatus. It has been discovered that the use of such a nebuliser in spray drying has a number of important advantages and these have not previously been recognised. The preferred USNs control the velocity of the particles and therefore the rate at which the particles are dried, which in turn affects the shape and density of the resultant particles. The use of USNs also provides an opportunity to perform spray drying on a larger scale than is possible using conventional spray drying apparatus with conventional types of nozzles used to create the droplets, such as 2-fluid nozzles.

The attractive characteristics of USNs for producing fine particle dry powders include: low spray velocity; the small amount of carrier gas required to operate the nebulisers; the comparatively small droplet size and narrow droplet size distribution produced; the simple nature of the USNs (the absence of moving parts which can wear, contamination, etc.); the ability to accurately control the gas flow around the droplets, thereby controlling the rate of drying; and the high output rate which makes the production of dry powders using USNs commercially viable in a way that is difficult and expensive when using a conventional two-fluid nozzle arrangement.

USNs do not separate the liquid into droplets by increasing the velocity of the liquid. Rather, the necessary energy is provided by the vibration caused by the ultrasonic nebuliser.

Further embodiments, may employ the use of ultrasonic nebulisers, rotary atomisers or electrohydrodynamic (EHD) atomizers to generate the particles.

In particular, it is disclosed that co-spray drying an active agent with an FCA under specific conditions can result in particles with excellent properties which perform extremely well when administered by a DPI for inhalation into the lung.

Spray drying may be used to produce the microparticles comprising the sumatriptan. The spray drying process may be adapted to produce spray-dried particles that include the active agent dispersed or suspended within a material that provides the controlled release properties.

Milling

The process of milling, may also be used to formulate the dry powder compositions according to the present invention. The manufacture of fine particles by milling can be achieved using conventional techniques. In the conventional use of the word, “milling” means the use of any mechanical process which applies sufficient force to the particles of active material that it is capable of breaking coarse particles (for example, particles with a MMAD greater than 100 μm) down to fine particles (for example, having a MMAD not more than 50 μm.). In the present invention, the term “milling” also refers to deagglomeration of particles in a formulation, with or without particle size reduction. The particles being milled may be large or fine prior to the milling step. A wide range of milling devices and conditions are suitable for use in the production of the compositions of the inventions. The selection of appropriate milling conditions, for example, intensity of milling and duration, to provide the required degree of force will be within the ability of the skilled person. The process of milling may also be used to formulate the dry powder compositions according to the present invention. The manufacture of fine particles by milling can be achieved using conventional techniques.

According to one embodiment of the invention, the active agent is milled with a force control agent and/or with an excipient material which can delay or control the release of the active agent when the active particles of the invention are deposited in the lung. Co-milling or co-micronising particles of active agent and particles of FCA or excipient will result in the FCA or excipient becoming deformed and being smeared over or fused to the surfaces of fine active particles. These resultant composite active particles comprising an FCA have been found to be less cohesive after the milling treatment.

The milling processes preferably apply a sufficient degree of force to break up tightly bound agglomerates of fine or ultra-fine particles, such that effective mixing and effective application of the additive material to the surfaces of those particles is achieved.

The additive material is preferably in the form of a coating on the surfaces of the particles of active material. The coating may be a discontinuous coating. The additive material may be in the form of particles adhering to the surfaces of the particles of active material.

At least some of the composite active particles may be in the form of agglomerates. However, when the composite active particles are included in a pharmaceutical composition, the additive material promotes the dispersal of the composite active particles on administration of that composition to a patient, via actuation of an inhaler.

The prior art mentions two types of processes in the context of co-milling or co-micronising active and additive particles.

Compressive Milling Processes In an alternative process for preparing the compositions according to the present invention, the powder components undergo a compressive milling process, such as processes termed mechanofusion (also known as ‘Mechanical Chemical Bonding’) and cyclomixing.

As the name suggests, mechanofusion is a dry coating process designed to mechanically fuse a first material onto a second material. It should be noted that the use of the terms “mechanofusion” and “mechanofused” are supposed to be interpreted as a reference to a particular type of milling process, but not a milling process performed in a particular apparatus. The compressive milling processes work according to a different principle to other milling techniques, relying on a particular interaction between an inner element and a vessel wall, and they are based on providing energy by a controlled and substantial compressive force. The process works particularly well where one of the materials is generally smaller and/or softer than the other.

The fine active particles and additive particles are fed into the vessel of a mechanofusion apparatus (such as a Mechano-Fusion system (Hosokawa Micron Ltd) or the Nobilta or Nanocular apparatus, where they are subject to a centrifugal force and are pressed against the vessel inner wall. The powder is compressed between the fixed clearance of the drum wall and a curved inner element with high relative speed between drum and element. The inner wall and the curved element together form a gap or nip in which the particles are pressed together. The principles behind these processes are distinct from those of alternative milling techniques in that they involve a particular interaction between an inner element and a vessel wall, and that these principles are based on providing energy by a controlled and substantial compressive force, preferably compression within a gap of predetermined width. As a result, the particles experience very high shear forces and very strong compressive stresses as they are trapped between the inner drum wall and the inner element (which has a greater curvature than the inner drum wall). The particles are pressed against each other with enough energy to locally heat and soften, break, distort, flatten and wrap the particles of one material (preferably the additive) around the core particle of the harder material (preferably the active material) to form a coating. The energy is generally sufficient to break up agglomerates and some degree of size reduction of both components may occur. However, in practice, this compression process produces little or no milling (i.e. size reduction) of the drug particles, especially where they are already in a micronised form (i.e. <10 μm), the only physical change which may be observed is a plastic deformation of the particles to a rounder shape.

The co-milling or co-micronising of active and additive particles may involve compressive type processes, such as mechanofusion, cyclomixing and related methods such as those involving the use of a Hybridiser or the Nobilta.

These mechanofusion and cyclomixing processes apply a high enough degree of force to separate the individual particles of active material and to break up tightly bound agglomerates of the active particles such that effective mixing and effective application of the additive material to the surfaces of those particles is achieved. An especially desirable aspect of the described co-milling processes is that the additive material becomes deformed in the milling and may be smeared over or fused to the surfaces of the active particles.

Jet-Milling

Jet mills are capable of reducing solids to particle sizes in the low-micron to submicron range. The grinding energy is created by gas streams from horizontal grinding air nozzles. Particles in the fluidized bed created by the gas streams are accelerated towards the centre of the mill, colliding with slower moving particles. The gas streams and the particles carried in them create a violent turbulence and as the particles collide with one another they are pulverized.

In the past, jet-milling has not been considered attractive for co-milling active and additive particles, with controlled compressive processes like Mechanical Chemical Bonding (mechanofusion) and cyclomixing being clearly preferred. The collisions between the particles in a jet mill are somewhat uncontrolled and those skilled in the art, therefore, considered it unlikely for this technique to be able to provide the desired deposition of a coating of additive material on the surface of the active particles. Moreover, it was believed that, unlike the situation with Mechanical Chemical Bonding and cyclomixing, segregation of the powder constituents occurred in jet mills, such that the finer particles, that were believed to often be the most desirable and effective, could escape from the process. In contrast, it could be clearly envisaged how techniques such as mechanofusion would result in the desired coating.

It should also be noted that it was also previously believed that the compressive or impact milling processes must be carried out in a closed system, in order to prevent segregation of the different particles. This has also been found to be untrue and the co-jet milling processes according to the present invention do not need to be carried out in a closed system. Even in an open system, the co-jet milling has surprisingly been found not to result in the loss of the small particles, even when using leucine as the additive material.

It has been discovered that composite particles of active and additive material can be produced by co-jet milling these materials. The resultant particles have excellent characteristics which lead to greatly improved performance when the particles are dispensed from a DPI for administration by inhalation. In particular, co-jet milling active and additive particles can lead to further significant particle size reduction. What is more, the composite active particles exhibit an enhanced FPD and FPF.

The effectiveness of the promotion of dispersal of active particles has been found to be enhanced by using the co-jet milling methods according to the present invention in comparison to compositions which are made by simple blending of similarly sized particles of active material with additive material. The phrase “simple blending” means blending or mixing using conventional tumble blenders or high shear mixing and basically the use of traditional mixing apparatus which would be available to the skilled person in a standard laboratory.

In another embodiment, the particles produced using the two-step process discussed above subsequently undergo mechanofusion. This final mechanofusion step is thought to “polish” the composite active particles, further rubbing the additive material into the particles. This allows one to enjoy the beneficial properties afforded to particles by mechanofusion, in combination with the very small particles sizes made possible by the co-jet milling.

In one embodiment, if required, the microparticles produced by the milling step can then be formulated with an additional excipient. This may be achieved by a spray drying process, e.g. co-spray drying. In this embodiment, the particles are suspended in a solvent and co-spray dried with a solution or suspension of the additional excipient. Preferred additional excipients include polysaccharides. Additional pharmaceutical effective excipients may also be used.

Jet-milling processes create high-energy impacts between media and particles or between particles. In practice, while these processes are good at making very small particles, it has been found that neither the ball mill nor the homogenizer were effective in producing dispersion improvements in resultant drug powders in the way observed for the compressive process. It is believed that these second impact processes are not as effective in producing a coating of additive material on each particle.

If a significant reduction in particle size is also required, co-jet milling is preferred, as disclosed in the earlier patent application published as WO 2005/025536. The co-jet milling process can result in composite active particles with low micron or sub-micron diameter, and these particles exhibit particularly good FPF and FPD, even when dispensed using a passive DPI.

Other Milling Procedures

Additionally, there are the impact milling processes involved in ball milling and the use of a homogenizer.

Ball milling is a suitable milling method for use in the prior art co-milling processes.

Centrifugal and planetary ball milling are especially preferred methods. Alternatively, a high pressure homogeniser may be used in which a fluid containing the particles is forced through a valve at high pressure producing conditions of high shear and turbulence. Such homogenisers may be more suitable than ball mills for use in large scale preparations of the composite active particles.

Suitable homogenisers include EmulsiFlex high pressure homogenisers which are capable of pressures up to 4000 bar, Niro Soavi high pressure homogenisers (capable of pressures up to 2000 bar), and Microfluidics Microfluidisers (maximum pressure 2750 bar). The milling step may, alternatively, involve a high energy media mill or an agitator bead mill, for example, the Netzsch high energy media mill, or the DYNO-mill (Willy A. Bachofen AG, Switzerland).

As discussed above, conventional methods comprising co-milling active material with additive materials (as described in WO 02/43701) result in composite active particles which are fine particles of active material with an amount of the additive material on their surfaces.

Shear forces on the particles, result in impactions between the particles and machine surfaces or other particles, and cavitation due to acceleration of the fluid which may all contribute to the fracture of the particles. Suitable homogenisers include the EmulsiFlex high pressure homogeniser, the Niro Soavi high pressure homogeniser and the Microfluidics Microfluidiser. The milling process can be used to provide the microparticles with mass median aerodynamic diameters as specified above.

Impact milling processes may be used to prepare compositions comprising triptans according to the present invention, with or without additive material. Such processes include ball milling and the use of a suitable homogenizer. Homogenisers may be more suitable than ball mills for use in large scale preparations of the composite active particles. In practice, while these processes are good at making very small particles, it has been found that neither the ball mill nor the homogenizer was particularly effective in producing dispersion improvements in resultant drug powders in the way observed for the compressive process. It is believed that the second impact processes are not as effective in producing a coating of additive material on each particle.

Milling Summary

Conventional methods comprising co-milling active material with additive materials (as described in WO 02/43701) result in composite active particles which are fine particles of active material with an amount of the additive material on their surfaces. The additive material is preferably in the form of a coating on the surfaces of the particles of active material. The coating may be a discontinuous coating. The additive material may be in the form of particles adhering to the surfaces of the particles of active material. Co-miffing or co-micronising particles of active agent and particles of additive (FCA) or excipient will result in the additive or excipient becoming deformed and being smeared over or fused to the surfaces of fine active particles, producing composite particles made up of both materials. These resultant composite active particles comprising an additive have been found to be less cohesive after the milling treatment.

At least some of the composite active particles may be in the form of agglomerates. However, when the composite active particles are included in a pharmaceutical composition, the additive material promotes the dispersal of the composite active particles on administration of that composition to a patient, via actuation of an inhaler.

Milling may also be carried out in the presence of a material which can delay or control the release of the active agent.

Where the compositions of the present invention include an additive material, the manner in which this is incorporated will have a significant impact on the effect that the additive material has on the powder performance, including the FPF and FPD.

High Shear Blending

Scaling up of pharmaceutical product manufacture often requires the use one piece of equipment to perform more than one function. An example of this is the use of a mixer-granulator which can both mix and granulate a product thereby removing the need to transfer the product between pieces of equipment. In so doing, the opportunity for powder segregation is minimised. High shear blending often uses a high-shear rotor/stator mixer (HSM), which has become used in mixing applications. Homogenizers or “high shear material processors” develop a high pressure on the material whereby the mixture is subsequently transported through a very fine orifice or comes into contact with acute angles. The flow through the chambers can be reverse flow or parallel flow depending on the material being processed. The number of chambers can be increased to achieve better performance. The orifice size or impact angle may also be changed for optimizing the particle size generated. Particle size reduction occurs due to the high shear generated by the high shear material processors while it passes through the orifice and the chambers. The ability to apply intense shear and shorten mixing cycles gives these mixers broad appeal for applications that require agglomerated powders to be evenly blended. Furthermore conventional HSMs may also be widely used for high intensity mixing, dispersion, disintegration, emulsification and homogenization.

It is well known to those skilled in the production of powder formulations that small particles, even with high-power, high-shear, mixers a relatively long period of “aging” is required to obtain complete dispersion, and this period is not shortened appreciably by increases in mixing power, or by increasing the speed of rotation of the stirrer so as to increase the shear velocity. High shear mixers can also be used if the auto-adhesive properties of the drug particles are so that high shear forces are required together with use of a force-controlling agent for forming a surface-energy-reducing particulate coating or film.

Dosing Regimen

Details of the therapy according to the present invention will depend on various factors, such as the age, sex or condition of the patient, and the existence or otherwise of one or more concomitant therapies. The nature and severity of the condition will also have to be taken into account.

In one embodiment, the composition provides a daily dose, which is the dose administered over a period of 24 hours, of between about 6 and about 60 mg. The daily doses will often be divided up into a number of doses. Preferably, the daily dose is between about 3 and about 40 mg. These daily doses may be administered at a single instance (usually involving multiple sequential inhalations), but it is expected that the daily dose will be spread out over the 24 hour period for patients experiencing prolonged migraine. In such cases, the patient may receive, on average, 2-3 separate single, or sets of sequential administrations, although some patients may receive 4-5 doses, or sets of sequential doses, with a daily extreme of, for example, 3 administrations of two sequential 10 mg doses, i.e. 60 mg in a 24 hour period.

In a yet further embodiment, the compositions according to the present invention are for use in providing treatment of the symptoms of migraine or for preventing the symptoms altogether. The patient is preferably able to administer a dose or a set of sequential doses and to ascertain within a period of no more than about 20 minutes, preferably no more than 15 minutes and most preferably within 10 minutes whether that administered dose, or a set of sequential doses, is sufficient to treat or prevent the symptoms of migraine. If a further dose, or set of sequential doses, is felt to be necessary, this may be safely administered and the procedure may be repeated until the desired therapeutic effect is achieved.

In another embodiment, the composition allows doses, or sets of sequential doses, to be administered at regular and frequent intervals, for example intervals of about 60 minutes, about 45 minutes, about 30 minutes, about 20 minutes, about 15 minutes or about 10 minutes, providing prophylactic therapy to avoid the patient experiencing migraine or migraine symptoms. In such an embodiment, the individual doses, or sets of sequential doses, administered at the chosen intervals will be adjusted to provide a daily dose within safe limits, whilst hopefully providing the patient with adequate relief from symptoms.

This self-titration of the triptan dose is possible as a result of the rapid onset of the therapeutic effect, the accurate and relatively small dose and the low incidence of side effects. It is also important that the mode of administration is painless and convenient, allowing repeated dosing without undue discomfort or inconvenience.

In one embodiment, the composition comprises a dose, or a set of sequential doses, of triptan to be administered to a patient of up to or of 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg or 30 mg. Preferably the dose is at least 1 mg, 2 mg, 3 mg or 4 mg.

Acute Migraine Therapy

In yet another embodiment, the doses of the triptan composition are to be administered to the patient as needed, that is, when the patient experiences or suspects the onset of migraine. This provides a pro renata or “on-demand” treatment. In this embodiment, a single effective dose, or set of sequential doses, of triptan may be administered where the amount of triptan in each such administration is preferably between 3 and 30 mg, more preferably between 4 and 25 mg and most preferably between 5 and 22 mg. Alternatively, multiple smaller doses, or sets of sequential doses, may be administered sequentially, with the effect of each dosing being assessed for efficacy by the patient before the next is administered. This allows for self-titration and optimisation of the dose when patients experience onset of migraine symptoms.

The severity of migraine attacks and the response to treatment may vary. Quite often patients may require only one drug for the majority of their attacks but on occasion several drugs may be required for more severe attacks. For the majority of patients, simply attending to the onset of migraine symptoms ensures quick relief. Provided the necessary contraindications have been considered, there are several important components for successful treatment. Therapy should be administered at the onset of headache. A variety of drug doses for treating such attacks include, for example, 900 mg of aspirin, 1000 mg of acetaminophen, 500-1000 mg of naproxen, 400-800 mg of ibuprofen, or a combination thereof.

In an alternate embodiment, the triptan, such as sumatriptan, may be combined with other acute treatments for migraine, for example non-steroidal anti-inflammatory drugs (NSAIDs) as in the case of Trexima™ (naproxen sodium, GlaxoSmithKline), simple analgesics, caffeine, opioids, barbiturate hypnotics and corticosteroids, calcitonin gene-related peptide (CGRP) antagonists, vanilloid agonists, glutamate modulators and nitric acid synthase inhibitors, or any combination thereof.

Preventative Migraine Therapy

In a preferred embodiment a combination of a triptan with prophylactic migraine drugs is provided. Such a combination would permit the patient to continue prophylaxis whilst treating a breakthrough migraine. Prophylactic migraine drugs may include for example, beta blockers, verapamil and pizotifen.

A variety of preventative therapies are in use with vary degrees of acceptability. Those that have a proven or well accepted use include the β-adrenergic-receptor antagonists (propranolol and metoprolol), amitriptyline, divalproex (valproate) and flunarizine. Serotonin antagonists such as pizotyline (pizotifen) and methysergide are also widely used. Verapamil and selective serotonin-reuptake inhibitors, whilst widely used, have still to provide evidence of real benefit. The final group of compounds that continue to show promise include gabapentin and topiramate.

In accordance with another embodiment of the present invention, a dose, or set of sequential doses, of sumatriptan is delivered to the lungs wherein said dose is sufficient to provide prophylaxis and/or therapeutic relief for acute mountain sickness and/or altitude headache preferably within 1 hour, more preferably within 30 minutes and most preferably within 10 minutes of administration.

In a further embodiment a combination of a triptan with a monoamine oxidase-A (MAO-A) inhibitor is disclosed, said combination being administered either simultaneously or sequentially, wherein said MAO-A is but not limited to, moclobemide, befloxatone, toloxatone, cimoxatone, amiflamine and harmaline wherein said combination can provide a reduced dose requirement for the triptan component and can provide a resultant increase in efficacy by increasing elimination half-life and reducing dosing frequency.

In a further embodiment, a composition can comprise a triptan administered simultaneously or sequentially with a non-steroidal anti-inflammatory drug (NSAID) or a Cox2 inhibitor such as celecoxib, piroxicam, meloxicam, mefenamic acid, flufenamic acid, flurbiprofen, naproxen, etodolac, aceclofenac or diflunisal.

In a further embodiment a composition comprising a triptan administered simultaneously or sequentially with an anaesthetic agent. Such a composition would comprise for example sumatriptan, frovatriptan, zolmitriptan, rizatriptan or naratriptan and an anaesthetic agent such as lidocaine, bupivacaine, ropivacaine, etidocaine or tetracaine. Additionally said composition may further comprise a beta blocker.

Further compositions may comprise a triptan administered simultaneously or sequentially with a cannabinoid, including CB1 and CB2 agonists, particularly dronabinol, nabilone and Sativex.

Further compositions may comprise a triptan administered simultaneously or sequentially with a Telmisartan and other angiotensin II receptor antagonists (ARB).

Further compositions may comprise a triptan administered simultaneously or sequentially with an N-methyl d-aspartate receptor (NMDAR) antagonist. The NMDA receptor antagonist may be selected from the group consisting of memantine, amantidine, rimantidine, ketamine, eliprodil, ifenprodil, dizocilpine, remacemide, iamotrigine, riluzole, aptiganel, phencyclidine, flupirtine, celfotel, felbamate, neramexane, spermine, spermidine, levemopamil, dextromethorphan, dextrorphan, and pharmaceutically acceptable salts thereof.

Further compositions may comprise a triptan administered simultaneously or sequentially with a 5-hydroxytryptamine-3 (5-HT₃) receptor antagonist that exhibits an anti-emetic action. The 5-HT₃ receptor antagonists or particular interest include dolasetron, granisetreon and ondansetron.

Simultaneous or sequential administration of a triptan with a dopamine antagonist for example domperidone, chlorpromazine or prochlorperazine is disclosed.

Simultaneous or sequential administration of a triptan with an antihistamine for example cyclizine or promethazine is disclosed.

Simultaneous or sequential administration of a triptan with a benzodiazepine for example lorazepam or midazolam is disclosed.

Furthermore triple combination therapies of a triptan and co-actives disclosed herein, for example the administration of a triptan, an anti-inflammatory with an anti-emetic to the pulmonary system is a preferential combination.

Pharmacokinetics

The combination of lung physiology and the attributes of the present invention result in rapid onset of action, a high degree of efficacy (pain relief at 2 hours in ≧70% patients), consistent systemic exposure which translate to a rapid and predictable therapeutic effect, in a form suitable for patients that are nauseous and/or vomiting and additionally avoiding the need for injections and their associated inconvenience.

Preferably, a Tₘₐₓ of as little as 15 minutes and more preferably less than 10 minutes is observed. The majority of patients achieved an onset of the therapeutic effect within 10 minutes following the inhalation of sumatriptan. Patients may expect a therapeutic effect as quickly as 4 or even 2 minutes after administration of the sumatriptan by pulmonary inhalation.

The concept of bioavailability within the desired time period is of therapeutic interest is paramount importance. When this is achieved, rapid therapeutic relief is ensured.

In a further embodiment of the present invention, the administration of the composition by pulmonary inhalation provides a dose dependent Cₘₐₓ.

In accordance with another embodiment of the present invention, a dose of sumatriptan is inhaled into the lungs and said dose is sufficient to provide a therapeutic effect in about 30 minutes or less. In some cases, the therapeutic effect is experienced within as little as about 20 minutes, more preferably less than about 15 minutes or even less than 10 minutes from administration.

In another embodiment of the invention, the administration of the composition by pulmonary inhalation provides a terminal elimination half-life of between 60 and 200 minutes.

In yet another embodiment, the administration of the composition by pulmonary inhalation provides a therapeutic effect with duration of at least 45 minutes, preferably at least 60 minutes. In a clinical trial, a mean duration of the therapeutic effect would be expected to be no different to subcutaneous administration.

According to one embodiment of the present invention, a composition comprising sumatriptan is provided, wherein the administration of the composition by pulmonary inhalation provides a Tₘₐₓ less than about 15 minutes and preferably within about 10 minutes of administration.

In one embodiment of the present invention, a Nominal Dose includes about 2 to about 10 mg of sumatriptan succinate, and the dose provides, in vivo, a mean Cₘₐₓ of from about 25 ng/ml to about 100 ng/ml. The Tₘₐₓ for any dose of sumatriptan occurs between 0.5 and 30 minutes after administration pulmonary inhalation, and preferably after between 1 and 15 minutes and most preferably between 2 and 10 minutes when measured via venous blood sampling. Importantly, the Cₘₐₓ obtained by arterial blood sampling is greater than approximately 1.5 times that observed from venous blood sampling as exemplified below. Furthermore, the arterial drug levels maintain the drug levels when compared to venous levels. The terminal elimination of the drug is approximately two hours for any dose. The elimination half life for a dose of sumatriptan delivered by pulmonary administration for the treatment of migraine as disclosed herein was approximately 95-191 minutes.

Thus, a composition comprising sumatriptan according to the present invention provides a Tₘₐₓ within 8 to 20 minutes of administration upon administration of the composition by pulmonary inhalation wherein the Cₘₐₓ is dose dependent. This rapid absorption of the sumatriptan upon inhalation should allow the administration of these compositions to provide a therapeutic effect in about 10 minutes or less.

The significance of these pharmacokinetics for the compositions of the present invention is that they show that inhalation of the sumatriptan compositions results in a consistent Tₘₐₓ of between 8 and 16 minutes with very little patient-to-patient variability. In particular, the range of Cₘₐₓ and CV are very similar to those seen following subcutaneous administration, and are less than those for oral and nasal administration.

A surprising observation for formulations of the present invention is the absence of an adverse effect on Forced Expiratory Volume in one second (FEV1). This is particularly surprising because pulmonary arteries and veins contain 5HT_{1B} and 5HT_{1A} receptors. These receptors are thought to be linked to the triptan-induced pulmonary vasoconstriction which manifest themselves as the triptan chest symptoms.

Delivery Devices

The inhalable compositions in accordance with the present invention are preferably administered via a dry powder inhaler (DPI), but can also be administered via a pressurized metered dose inhaler (pMDI), or even via a nebulised system.

Dry Powder Inhalers

The compositions according to the present invention may be administered using active or passive DPIs. As it has now been identified how one may tailor a dry powder formulation to the specific type of device used to dispense it, this means that the perceived disadvantages of passive devices where high performance is sought may be overcome.

Preferably, these FPFs are achieved when the composition is dispensed using an active DPI, although such good FPFs may also be achieved using passive DPIs, especially where the device is one as described in the earlier patent application published as WO 2005/037353 and/or the dry powder composition has been formulated specifically for administration by a passive device.

In one embodiment of the invention, the DPI is an active device, in which a source of compressed gas or alternative energy source is used. Examples of suitable active devices include Aspirair™ (Vectura) and the active inhaler device produced by Nektar Therapeutics (as disclosed in U.S. Pat. No. 6,257,233), and the ultrasonic Microdose™ or Oriel™ devices.

In an alternative embodiment, the DPI is a passive device, in which the patient's breath is the only source of gas which provides a motive force in the device. Examples of “passive” dry powder inhaler devices include the Rotahaler™ and Diskhaler™ (GlaxoSmithKline) and the Turbohaler™ (Astra-Draco) and Novolizer™ (Viatris GmbH) and GyroHaler™ (Vectura).

The dry powder formulations may be pre-metered and kept in capsules or foil blisters which offer chemical and physical protection whilst not being detrimental to the overall performance. Alternatively, the dry powder formulations may be held in a reservoir-based device and metered on actuation. Examples of “reservoir-based” inhaler devices include the Clickhaler™ (Innovata) and Duohaler™ (Innovata), and the Turbohaler™ (Astra-Draco). Actuation of such reservoir-based inhaler devices can comprise passive actuation, wherein the patient's breath is the only source of energy which generates a motive force in the device.

In a dry powder inhaler, the dose to be administered is stored in the form of a non-pressurized dry powder and, on actuation of the inhaler, the particles of the powder are expelled from the device in the form of a cloud of finely dispersed particles that may be inhaled by the patient.

Dry powder inhalers can be “passive” devices in which the patient's breath is the only source of gas which provides a motive force in the device. Examples of “passive” dry powder inhaler devices include the Rotahaler and Diskhaler (GlaxoSmithKline), the Monohaler (MIAT), the Gyrohaler (trademark) (Vectura) the Turbohaler (Astra-Draco) and Novolizer (trade mark) (Viatris GmbH). Alternatively, “active” devices may be used, in which a source of compressed gas or alternative energy source is used. Examples of suitable active devices include Aspirair (trade mark) (Vectura Ltd) and the active inhaler device produced by Nektar Therapeutics (as covered by U.S. Pat. No. 6,257,233).

It is generally considered that different compositions perform differently when dispensed using passive and active type inhalers. Passive devices create less turbulence within the device and the powder particles are moving more slowly when they leave the device. This leads to some of the metered dose remaining in the device and, depending on the nature of the composition, less deagglometation upon actuation. However, when the slow moving cloud is inhaled, less deposition in the throat is often observed. In contrast, active devices create more turbulence when they are activated. This results in more of the metered dose being extracted from the blister or capsule and better deagglomeration as the powder is subjected to greater shear forces. However, the particles leave the device moving faster than with passive devices and this can lead to an increase in throat deposition.

It has been surprisingly found that the compositions of the present invention with their high proportion of sumatriptan perform well when dispensed using both active and passive devices. Whilst there tends to be some loss along the lines predicted above with the different types of inhaler devices, this loss is minimal and still allows a substantial proportion of the metered dose of sumatriptan to be deposited in the lung. Once it reaches the lung, the sumatriptan is rapidly absorbed and exhibits consistent absorption and higher bioavailability than oral or nasal sumatriptan formulations.

Particularly preferred “active” dry powder inhalers are referred to herein as Aspitair® inhalers and are described in more detail in WO 01/00262, WO 02/07805, WO 02/89880 and WO 02/89881, the contents of which are hereby incorporated by reference. It should be appreciated, however, that the compositions of the present invention can be administered with either passive or active inhaler devices.

Other Inhalers

In a yet further embodiment, the compositions are dispensed using a pressurised metered dose inhaler (pMDI), a nebuliser or a soft mist inhaler. Drug doses delivered by pressurised metered dose inhalers tend to be of the order of 1 μg to 3 mg. Examples of suitable devices include pMDIs such as Modulite® (Chiesi), SkyeFine™ and SkyeDry™ (SkyePharma). Nebulisers such as Porta-Neb®, Inquaneb™ (Pari) and Aquilon™, and soft mist inhalers such as eFlow™ (Pari), Aerodose™ (Aerogen), Respimat® Inhaler (Boehringer Ingelheim GmbH), AERx® Inhaler (Aradigm) and Mystic™ (Ventaira Pharmaceuticals, Inc.).

Compositions suitable for use in these devised include solutions and suspensions, both of which may be dispensed using a pressurised metered dose inhaler (pMDI). The pMDI compositions according to the invention can comprise the dry powder composition discussed above, mixed with or dissolved in a liquid propellant.

In one embodiment, the propellant is CFC-12 or an ozone-friendly, non-CFC propellant, such as 1,1,1,2-tetrafluoroethane (HFC 134a), 1,1,1,2,3,3,3-heptafluoropropane (HFC-227), HCFC-22 (difluororchloromethane), HFA-152 (difluoroethane and isobutene) or combinations thereof. Such formulations may require the inclusion of a polar surfactant such as polyethylene glycol, diethylene glycol monoethyl ether, polyoxyethylene sorbitan monolaurate, polyoxyethylene sorbitan monooleate, propoxylated polyethylene glycol, and polyoxyethylene lauryl ether for suspending, solubilising, wetting and emulsifying the active agent and/or other components, and for lubricating the valve components of the pMDI.

Conclusion

In conclusion, the advantages of pulmonary delivery may be summarised as follows. The rapid onset of action, a high degree of efficacy, increased delivery efficiency resulting in consistent systemic exposure translates into a rapid and predictable therapeutic effect. The excellent bioavailability achieved by pulmonary delivery from a dosage level lends further support for future use of this route of administration. The delivery of a rapid acting dosage form that avoids bad taste is particularly suitable for patients that are either nauseous and/or vomiting. Furthermore, a route of administration that also avoids the need for injections at a time when patients are unlikely to want to self administer medication must be viewed as more patient friendly.

Pulmonary delivery via oral inhalation, not being subject to some of the complexities surrounding nasal administration, results in more rapid and consistent systemic exposure which translates to an accelerated and predictable therapeutic response. These parameters are key unmet clinical needs when considering the treatment of many disorders of the central nervous system, and migraine in particular.

General Statement

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific compositions and methods described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or “an” when used in conjunction with the term “comprising” in the claims and/or the specification may mean “one”, but it is also consistent with the meaning of “one or more”, “at least one”, and “one or more than one”. The use of the term “or” in the claims is used to mean “and/or” unless explicitly indicated to refer to alternatives only or the alternatives are mutually exclusive, although the disclosure supports a definition that refers to only alternatives and “and/or”. Throughout this application, the term “about” is used to indicate that a value includes the inherent variation of error for the device, the method being employed to determine the value, or the variation that exists among the study subjects.

As used in this specification, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, BBC, AAABCCCC, CBBAAA, CABABB and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### EXAMPLES

### Example 1

### Spray Dried Sumatriptan

Prior to commencing spray drying, a sample of sumatriptan succinate raw material was analysed using Differential Scanning Calorimetry (DSC) to determine the glass transition temperature (Tg). The Tg was established to be 46.5° C. with a melting point of 167° C. This has an impact on the spray drying process parameters as the drying temperatures must be chosen to prevent the exposure of the active to temperatures above 46.5° C. A sumatriptan succinate in water formulation (2% w/v) was spray dried on the mini spray dryer even with low outlet temperatures, no powder was collected and a glassy coating was observed on all surfaces.

In order to increase the Tg of the formulation, trehalose and leucine were added in different ratios. A number of formulations were prepared, the following tables summarise the data obtained for the three successful formulations.

In Vitro Data

TABLE 1
Particle Size Results

| Batch Number | Run | X₁₀ μm | X₅₀ μm | X₉₀ μm |
|---|---|---|---|---|
| Sumatriptan:trehalose:leucine | 1 | 0.77 | 2.08 | 58.24 |
| (50:45:5% w/w/w) | 2 | 0.72 | 2.01 | 56.66 |
| | 3 | 0.78 | 1.98 | 47.38 |
| | Mean | 0.76 | 2.02 | 54.09 |
| Sumatriptan:trehalose | 1 | 0.61 | 1.34 | 8.61 |
| (15:85% w/w) | 2 | 0.64 | 1.34 | 3.33 |
| | 3 | 0.66 | 1.33 | 2.89 |
| | Mean | 0.64 | 1.34 | 4.94 |
| Sumatriptan:trehalose | 1 | 0.80 | 1.84 | 39.45 |
| (30:70% w/w) | 2 | 0.71 | 1.47 | 3.40 |
| | 3 | 0.73 | 1.54 | 3.73 |
| | Mean | 0.75 | 1.62 | 15.53 |

Aerosol Performance Testing

Numbers in bold are mean values. All values relate to sumatriptan succinate. The formulations were hand filled into hydroxypropyl methylcellulose (HMPC) capsules (all doses filled to equate to 9 mg sumatriptan succinate in the capsule) and fired from a Monohaler® (90 l/min).

TABLE 2
Aerosol Performance Testing Results

| | | Fine | Fine | Fine | Fine |
|---|---|---|---|---|---|
| | | Particle | Particle | Particle | Particle |
| | Delivered | Mass μg | Fraction % | Mass μg | Fraction % |
| Batch Number | Dose μg | (<3.3 μm) | (<3.3 μm) | (<5.8 μm) | (<5.8 μm) |
| Sumatriptan:trehalose: | 8400 | 3440 | 40.9% | 5170 | 61.6% |
| leucine (50:45:5% w/w/w) | 7790 | 3440 | 44.2% | 4930 | 63.3% |
| | 810 | 344 | 42.6% | 505 | 62.5% |
| Sumatriptan:trehalose | 3960* | 1870 | 47.1% | 2630 | 66.3% |
| (15:85% w/w) | 6070 | 3120 | 51.4% | 4050 | 66.7% |
| | 502 | 250 | 49.3% | 334 | 66.5% |
| Sumatriptan:trehalose | 7600 | 3300 | 43.4% | 4790 | 63.1% |
| (30:70% w/w) | 7010 | 3440 | 49.1% | 4720 | 67.4% |
| | 731 | 337 | 46.3% | 476 | 65.3% |

*Thought to be due error in weight of material added to capsule

### Example 2

### Spray Dried Frovatriptan

An evaluation of inhaled frovatriptan formulations was performed. A spray drying process for frovatriptan was identified for producing particles suitable for systemic pulmonary delivery. In addition, a frovatriptan:magnesium stearate formulation (95:5% w/w) was prepared using the mechanofusion process as described previously.

Formulation Methodology

Four frovatriptan formulations were generated; the batch details are as follows:

1. Spray dried 100% frovatriptan

2. Spray dried frovatriptan:trehalose (50:50% w/w)

3. Spray dried frovatriptan:trehalose (75:25% w/w)

4. Frovatriptan:MgSt (90:10% w/w)

Each feedstock was spray dried using the spray drying parameters shown in the table below.

TABLE 3
Operating parameters for spray dried frovatriptan formulations

| Process parameter | Set point |
|---|---|
| Inlet temperature (° C.) | 130 |
| Outlet temperature (° C.) | >75 |
| Atomisation pressure (bar) | 4 |
| Atomisation flow rate (L/min) | 30 |
| Drying airflow pressure (L/sec) | 4.5 |
| Solution feed rate (g/min) | 5 |

The following methodologies were used to evaluate the four frovatriptan formulations.

In Vitro Methodology

Particle Size Analysis

Pre-commencement checks and set-up of the Sympatec were performed. A standard reference analysis was performed in triplicate to check the performance of the R2 lens by placing 10 to 20 mg of the SiC reference material onto the vibri feeder tray. A small amount (10 to 20 mg) of frovatriptan formulation was placed at the end of the vibri feeder tray and a reference measurement performed. Once this was complete the measurement of the frovatriptan formulation was performed. This procedure was repeated in triplicate and data recorded.

Karl Fischer Moisture Analysis

Pre-commencement checks and standardisation of the Karl Fischer instrument were performed. The appropriate test method was selected and loaded. All relevant details for sample analysis were stored on the touch control panel detail window.

200 mg of frovatriptan formulation was accurately weighed into a glass weighing scoop boat and the balance tared. The start button on the Metrohm 841 control panel was pressed to begin neutralisation of the titrating vessel; once the instrument drift was stable the control panel displayed ‘conditioning OK’. The start button was pressed again and automatically a 60 second countdown began. The weighed sample was added to the titration vessel within the 60 second window, ensuring that the sample was dispensed into the solution and none was left on the vessel wall or probe. The empty glass weighing scoop boat was reweighed and the negative mass displayed on the balance was entered onto the control panel when prompted and the continue button pressed immediately after.

Once the titration was complete and the titration end point reached the display showed ‘determination being finished’. The analysis result was generated and a hard copy printed.

This procedure was repeated in triplicate. Upon completion of three determinations the ‘statistics’ option and then ‘statistics overview’ was selected on the control panel, which gave a hard copy printout of the titer determination with individual values and relative standard deviation.

Andersen Cascade Impactor

Pre-commencement checks and set up for ACI analysis were performed. An empty HPMC capsule was weighed on a 6 place analytical balance and the weight recorded as W1. A known quantity (mg) of spray dried frovatriptan formulation was added to the capsule and reweighed and recorded as W2. The capsule was inserted into the Monohaler® device and closed. The Monohaler® device was inserted into the mouthpiece adaptor, ensuring that the vacuum pump was running and the two way solenoid valve was closed. The Monohaler® was activated for 2.67 seconds.

Once the solenoid valve was closed the Monohaler® was removed from the ACI. The capsule was carefully removed, reweighed and recorded as W3. The amount of powder cleared from the capsule was determined. The impactor samples were prepared and analysed using the methodology outlined in a laboratory notebook.

Visual Appearance

Visual observations of the spray dried frovatriptan formulations were made for appearance in terms of powder flow characteristics and colour of the resulting material.

In Vitro Data

Each of the frovatriptan formulations was evaluated to determine the particle size, moisture content and aerosol performance from the Monohaler® device.

TABLE 4
Particle size for frovatriptan formulations

| | Run | X50 μm | X90 μm |
|---|---|---|---|
| Spray dried 100% frovatriptan | 1 | 1.2 | 2.3 |
| | 2 | 1.3 | 3.4 |
| | 3 | 1.3 | 4.8 |
| | Mean | 1.3 | 3.5 |
| Spray dried frovatriptan:trehalose | 1 | 1.3 | 57.4 |
| (75:25% w/w) | 2 | 1.1 | 2.0 |
| | 3 | 1.1 | 2.0 |
| | Mean | 1.2 | 2.0 |
| Spray dried frovatriptan:trehalose | 1 | 1.2 | 3.0 |
| (50:50% w/w) | 2 | 1.2 | 2.1 |
| | 3 | 1.1 | 2.1 |
| | Mean | 1.2 | 2.4 |
| Frovatriptan:MgSt (95:5% w/w) | 1 | 1.7 | 3.0 |
| | 2 | 1.7 | 3.0 |
| | 3 | 1.7 | 3.0 |
| | Mean | 1.7 | 3.0 |

TABLE 5
Moisture Content

| | | Moisture |
|---|---|---|
| | Run | Content % |
| Spray dried 100% frovatriptan | 1 | 3.6 |
| | 2 | 3.5 |
| | 3 | 3.5 |
| | Mean | 3.6 |
| Spray dried frovatriptan:trehalose | 1 | 3.3 |
| (75:25% w/w) | 2 | 3.4 |
| | 3 | 3.4 |
| | Mean | 3.4 |
| Spray dried frovatriptan:trehalose | 1 | 3.4 |
| (50:50% w/w) | 2 | 3.2 |
| | 3 | 3.3 |
| | Mean | 3.3 |
| Frovatriptan:MgSt | 1 | 5.9 |
| (95:5% w/w) | 2 | 5.7 |
| | 3 | 5.8 |
| | Mean | 5.8 |

TABLE 6
ACI Performance

| | Formulation | | | | | |
|---|---|---|---|---|---|---|
| | FPM μg | FPF % | FPM μg | FPF % | FPM μg | FPF % |
| | <3.3 μm | <3.3 μm | <5.0 μm | <5.0 μm | <5.8 μm | <3.8 μm |
| Spray dried | 4050 | 61 | 4610 | 70 | 4760 | 72 |
| 100% | 3240 | 53 | 3680 | 60 | 3800 | 62 |
| frovatriptan | 3645 | 57 | 4145 | 65 | 4280 | 67 |
| Spray dried | 4160 | 57 | 4660 | 64 | 4790 | 66 |
| frovatriptan: | 3920 | 64 | 4530 | 74 | 4660 | 77 |
| trehalose | 4040 | 61 | 4595 | 69 | 4725 | 72 |
| (75:25% w/w) | | | | | | |
| Spray dried | 4090 | 64 | 4660 | 73 | 4800 | 75 |
| frovatriptan: | 4050 | 55 | 4590 | 63 | 4720 | 64 |
| trehalose | 4070 | 60 | 4625 | 68 | 4760 | 70 |
| (50:50% w/w) | | | | | | |
| Frovatriptan: | 3940 | 67 | 4860 | 82 | 5070 | 86 |
| MgSt | 4210 | 69 | 5250 | 86 | 5420 | 89 |
| (95:5% w/w) | 4075 | 68 | 5055 | 84 | 5245 | 88 |

The formulations have a similar deposition <3.3 μm which is 43-51% of the nominal dose.

TABLE 7
Visual Appearance

| Formulation | Description |
|---|---|
| 100% spray dried | White coloured powder |
| frovatriptan | |
| 75:25% w/w spray dried | White to off white coloured powder |
| frovatriptan:trehalose | |
| 50:50% w/w spray dried | White to off white coloured powder |
| frovatriptan:trehalose | |
| 95:5% w/w Mechanofused | White to off white coloured powder |
| frovatriptan:MgSt | |

The four frovatriptan formulation feed stocks used for spray drying; frovatriptan (100% w/w), frovatriptan:trehalose (50:50% w/w), frovatriptan:trehalose (75:25% w/w) and frovatriptan:magnesium stearate (MgSt) (95:5% w/w) readily dissolved in purified water to give stable solutions and no formulation issues were encountered.

The frovattiptan:trehalose (75:25% w/w) particle size of run 1 was skewed by large agglomerate and the frovatriptan:MgSt (95:5% w/w) moisture content result was above the acceptance criteria however this does not affect the performance characteristics at this stage.

There were no unexpected visual observations for any of the spray dried frovatriptan formulation batches. All of the spray dried powder was either of white colouration for the 100% spray dried frovatriptan batches or white to off white colouration for the frovatriptan:trehalose batches.

All formulations cleared well from the Monohaler™ device.

The frovatriptan formulations were found to have comparable particle size, moisture and aerosol performance characteristics. The fine particle mass data obtained for all formulations was found to be appropriate for systemic delivery. In summary, data generated for the frovatriptan formulations provides confidence that a suitable pulmonary formulation was developed.

### Example 3

### Jet-Milled Sumatriptan Succinate (95%) with Magnesium Stearate (5%) which was then Mechanofused

An evaluation of inhaled sumatriptan formulations was performed. A jet-milled then mechanofused process for sumatriptan was identified for producing particles suitable for systemic pulmonary delivery.

Formulation Methodology

Sumatriptan succinate (800 g) and magnesium stearate (40 g) were pre-mixed using the Turbula mixer for 10 minutes at 30 rpm then allowed to rest for 10 minutes. The particles were then co-jet milled in a Hosokawa Alpine spiral jet mill (100AS) to produce a particle d50 (particle size analysis by Malvern Mastersizer dry cell analysis) below 2.2 μm (preferably d50 below 1.5 μm). The formulation was prepared using parameters shown in the table below.

TABLE 8
Operating parameters for Hosokawa Alpine spiral jet mill (100AS)

| 100AS parameters | Set point | |
|---|---|---|
| Gas | Nitrogen | |
| Venturi | 8-10 | bar |
| Grinding | 5-7 | bar |
| Feed rate | 15-25 | g/min |

The mechanofusion system used was a Hosokawa Micron ‘Mini Kit’. The particles were added to the mechanofusion system (Hosokawa Micron ‘Mini Kit’ with a 3 mm rotor gap size) in sub-batch sizes of 30-40 g with the system running in the region of 250 rpm. The particles were then pre-mixed in the mechanofusion system for 5 minutes (mixing speed in the region of 1000 rpm) then the particles were mechanofused for 10 minutes (mixing speed in the region of 4000 rpm). The generated sub-batches were combined by mixing in a Turbula mixer for 5 minutes at 30 rpm to produce a final formulation.

Formulation Blending

Unprocessed Lactose (LH200)

Lactose (LH200) was obtained from FreislandCampina (FrieslandDomo) and processed by blending with the additive and API to create formulations of up to approximately 4000 g.

It will be appreciated that various types of lactose may be used, for example Lactochem® Extra Fine or Respitose® SV003 (DMV International) and ranges of lactose may vary from about 5-80% (w/w) of the total formulation.

Processed Lactose (Mechano-Chemical Bonding)

Lactose (LH200) was obtained from FreislandCampina (FrieslandDomo) and processed by blending with the additive and API before samples of up to approximately 4000 g were jet-milled into final formulations for processing by mechano-chemical bonding.

Additional processes omitted the jet-miffing stages before undergoing processing by mechano-chemical bonding. It will be appreciated that various types of lactose may be used, for example Lactochem® Extra Fine or Respitose® SV003 (DMV International) and ranges of lactose may vary from about 5-80% (w/w) of the total formulation.

In Vitro Methodology

Bulk and Tapped Density

Bulk and tapped densities were determined based on USP30 (2007) section 616. Tapped density were determined using the Stampfvolumeter Stav2003 with a 10 ml measuring cylinder. 10 ml was placed into a measuring cylinder were then placed onto the Stampfvolumeter and tapped in sets of 500 taps until a constant volume measurement was obtained between two sets of taps. The volume measurements were then used to calculate densities, Carr's Index using the equations below.

Bulk Density (g/ml)=(weight of drug)/(volume of drug)

Tapped density (g/ml)=(weight of tapped drug)/(volume of tapped drug)

Carr's index (%)=100×(Tapped density−bulk density)/(Tapped density)

Andersen Cascade Impactor

Pre-commencement checks and set up for ACI analysis were performed. An empty HPMC capsule was weighed on a 5 place analytical balance and the weight recorded. A known quantity (mg) of formulation was added to the capsule and reweighed and recorded. The capsule was inserted into the Monohaler® device and closed. The Monohaler® device was inserted into the mouthpiece adaptor, ensuring that the vacuum pump was running and the two way solenoid valve was closed. The Monohaler® was activated for 2.67 seconds by opening the solenoid valve thereby ensuring 4 L or air was drawn through the device at 90 L/min.

Once the solenoid valve was closed the Monohaler® was removed from the ACI. The filled capsule and device were pre-weighed, and reweighed upon completion of the assessment. The amount of powder cleared from the capsule was determined. Five capsules were fired into an ACI prior to disassembly. The impactor samples were prepared and analysed by suitable UV methodology.

In Vitro Data

TABLE 9
Tapped Density Results
Jet-milled then MCB Formulations

| | | | | | |
|---|---|---|---|---|---|
| Sumatriptan | 70% | 70% | 90% | 95% | 97.5% |
| FCA | 10% MgSt | 5% MgSt | 10% MgSt | 5% MgSt | 2.5% MgSt |
| Lactose | 20% LH200 | 25% LH200 | 0% | 0% | 0% |
| Bulk Density | 0.34 | 0.34 | 0.28 | 0.32 | 0.32 |
| Tapped Density | 0.59 | 0.60 | 0.47 | 0.45 | 0.47 |
| Carr's Index (%) | 43 | 43 | 41 | 33 | 32 |

TABLE 10
Tapped Density Results
Jet Milled Formulations

| | | | |
|---|---|---|---|
| Sumatriptan | 90% | 90% | 90% |
| FCA | 10% Leucine | 10% MgSt | 10% Leucine |
| Lactose | 0% | 0% | 0% |
| Bulk Density | 0.13 | 0.17 | 0.13 |
| Tapped Density | 0.22 | 0.29 | 0.22 |
| Cart's Index (%) | 39 | 42 | 39 |

TABLE 11
Tapped Density Results
Jet Milled Formulations

| | | | |
|---|---|---|---|
| Sumatriptan | 98% | 95% | 90% |
| FCA | 2% MgSt | 5% MgSt | 10% MgSt |
| Lactose | 0% | 0% | 0% |
| Bulk Density | 0.15 | 0.16 | 0.17 |
| Tapped Density | 0.25 | 0.27 | 0.29 |
| Carr's Index (%) | 40 | 40 | 42 |

TABLE 12
Summary of Drug Delivery Performance - Size 3 HPMC
Capsules, Blister Packed. Test Device: Monohaler.

| | Sumatriptan Base 2 mg Inhalation Powder | | | | | |
|---|---|---|---|---|---|---|
| | 25° C./60% RH | | | | | |
| | Initial | 1 month | 3 months | 6 months | 9 months | 12 months |
| Nominal Dose (mg) | 2 | 2 | 2 | 2 | 2 | 2 |
| Delivered Dose (mg) | 1.6-2.2 | 1.7-2.0 | 1.7-2.0 | 1.7-2.2 | 1.7-2.1 | 1.3-1.9 |
| FPD ≦5 μm (mg) | 1.6-2.0 | 1.4-2.0 | 1.5-1.8 | 1.6-1.8 | 1.4-1.7 | 1.6-1.7 |
| FPF ≦5 μm (%) | 78-89 | 64-91 | 88-89 | 86-89 | 72-88 | 78-87 |
| FPD ≦3 μm (mg) | N/A | 1.2-1.7 | 1.3-1.6 | 1.4-1.5 | 0.1-1.4 | 1.3-1.4 |
| FPF ≦3 μm (%) | N/A | 54-79 | 74-77 | 73-76 | 51-76 | 65-70 |
| MMAD (μm) | 2.0-2.1 | 2.0-2.1 | 2.0-2.1 | 2.0-2.1 | 2.1-2.2 | 2.1-2.2 |

TABLE 13
Summary of Drug Delivery Performance, Size 3 HPMC
Capsules, Blister Packed. Test Device: Monohaler.

| | Sumatriptan Base 2 mg Inhalation Powder | | | |
|---|---|---|---|---|
| | 40° C./75% RH | | | |
| | Initial | 1 month | 3 months | 6 months |
| Nominal Dose (mg) | 2 | 2 | 2 | 2 |
| Delivered Dose (mg) | 1.6-2.2 | 1.4-1.9 | 1.2-1.8 | 1.4-2.4 |
| FPD ≦5 μm (mg) | 1.6-2.0 | 1.5-1.6 | 1.4-1.5 | 1.3-1.6 |
| FPF ≦5 μm (%) | 78-89 | 81-87 | 81-86 | 62-86 |
| FPD ≦3 μm (mg) | N/A | 1.2-1.3 | 1.1-1.3 | 0.9-1.3 |
| FPF ≦3 μm (%) | N/A | 65-71 | 64-70 | 44-69 |
| MMAD (μm) | 2.0-2.1 | 2.2-2.3 | 2.1-2.3 | 2.2-2.5 |

TABLE 14
Summary of Drug Delivery Performance, Size 3 HPMC
Capsules, Blister Packed. Test Device: Monohaler.

| | Sumatriptan Base 2 mg Inhalation Powder | | |
|---|---|---|---|
| | 25° C./60% RH | | |
| | Initial | 1 month | 3 months |
| Nominal Dose (mg) | 2 | 2 | 2 |
| Delivered Dose (mg) | 1.6-2.2 | 1.7-2.1 | 1.7-2.0 |
| FPD ≦5 μm (mg) | 1.7-1.8 | 1.6-1.7 | 1.6-1.7 |
| FPF ≦5 μm (%) | 77-82 | 84-86 | 84-86 |
| FPD ≦3 μm (mg) | 1.5-1.6 | 1.5 | 1.4 |
| FPF ≦3 μm (%) | 69-73 | 74-77 | 72-75 |
| MMAD (μm) | 2.0 | 1.9-2.0 | 2.0-2.1 |

TABLE 15
Summary of Drug Delivery Performance, Size 3 HPMC
Capsules, Blister Packed. Test Device: Monohaler.

| | Sumatriptan Base 2 mg Inhalation Powder | | |
|---|---|---|---|
| | 30° C./65% RH | | |
| | Initial | 1 month | 3 months |
| Nominal Dose (mg) | 2 | 2 | 2 |
| Delivered Dose (mg) | 1.6-2.2 | 1.6-1.9 | 1.7-2.3 |
| FPD ≦5 μm (mg) | 1.7-1.8 | 1.4-1.7 | 1.4-1.7 |
| FPF ≦5 μm (%) | 77-82 | 83-87 | 75-84 |
| FPD ≦3 μm (mg) | 1.5-1.6 | 1.3-1.5 | 1.3-1.4 |
| FPF ≦3 μm (%) | 69-73 | 74-77 | 66-74 |
| MMAD (μm) | 2.0 | 1.9-2.0 | 2.0 |

TABLE 16
Summary of Drug Delivery Performance, Size 3 HPMC
Capsules, Blister Packed. Test Device: Monohaler.

| | Sumatriptan Base 2 mg Inhalation Powder | | |
|---|---|---|---|
| | 40° C./75% RH | | |
| | Initial | 1 month | 3 months |
| Nominal Dose (mg) | 2 | 2 | 2 |
| Delivered Dose (mg) | 1.6-2.2 | 1.5-1.9 | 1.6-1.8 |
| FPD ≦5 μm (mg) | 1.7-1.8 | 1.1-1.6 | 1.4-1.6 |
| FPF ≦5 μm (%) | 77-82 | 66-87 | 76-83 |
| FPD ≦3 μm (mg) | 1.5-1.6 | 0.9-1.4 | 1.3 |
| FPF ≦3 μm (%) | 69-73 | 56-77 | 65-72 |
| MMAD (μm) | 2.0 | 2.0-2.1 | 1.9-2.1 |

TABLE 17
Summary of Drug Delivery Performance, Size 3 HPMC
Capsules, Blister Packed. Test Device: Monohaler.

| | Sumatriptan Base 5 mg Inhalation Powder | | | | | |
|---|---|---|---|---|---|---|
| | 25° C./60% RH | | | | | |
| | Initial | 1 month | 3 months | 6 months | 9 months | 12 months |
| Nominal Dose (mg) | 5 | 5 | 5 | 5 | 5 | 5 |
| Delivered Dose (mg) | 4.3-4.8 | 4.2-4.8 | 4.1-4.7 | 3.4-4.4 | 4.1-4.8 | 3.7-4.6 |
| FPD ≦5 μm (mg) | 3.2-3.7 | 3.1-3.5 | 3.3-3.7 | 3.3-3.6 | 3.3-3.6 | 2.9-3.5 |
| FPF ≦5 μm (%) | 70-80 | 74-78 | 77-78 | 74-81 | 73-82 | 72-79 |
| FPD ≦3 μm (mg) | 2.7-3.2 | 2.6-2.9 | 2.8-3.1 | 2.7-3.0 | 2.9-3.1 | 2.4-2.9 |
| FPF ≦3 μm (%) | 59-68 | 62-66 | 65-66 | 61-69 | 64-71 | 60-64 |
| MMAD (μm) | 2.1 | 2.1-2.2 | 2.1-2.2 | 1.5 | 2.0 | 2.1-2.2 |

TABLE 18
Summary of Drug Delivery Performance, Size 3 HPMC
Capsules, Blister Packed. Test Device: Monohaler.

| | Sumatriptan Base 5 mg Inhalation Powder | | | | | |
|---|---|---|---|---|---|---|
| | 30° C./65% RH | | | | | |
| | Initial | 1 month | 3 months | 6 months | 9 months | 12 months |
| Nominal Dose (mg) | 5 | 5 | 5 | 5 | 5 | 5 |
| Delivered Dose (mg) | 4.3-4.8 | 4.1-4.6 | 3.7-4.5 | 4.1-4.5 | 4.0-4.6 | 3.3-4.9 |
| FPD ≦5 μm (mg) | 3.2-3.7 | 2.9-3.4 | 3.5-4.0 | 3.1-3.4 | 3.4-3.5 | 3.2-3.6 |
| FPF ≦5 μm (%) | 70-80 | 67-77 | 81-85 | 71-76 | 76-83 | 74-80 |
| FPD ≦3 μm (mg) | 2.7-3.2 | 2.5-2.9 | 3.1-3.4 | 2.5-2.9 | 2.8-3.1 | 2.8-3.1 |
| FPF ≦3 μm (%) | 59-68 | 59-65 | 71-73 | 58-66 | 64-72 | 65-68 |
| MMAD (μm) | 2.1 | 2.1 | 2.0-2.1 | 2.1-2.2 | 2.0-2.1 | 2.1-2.2 |

TABLE 19
Summary of Drug Delivery Performance, Size 3 HPMC
Capsules, Blister Packed. Test Device: Monohaler.

| | Sumatriptan Base 5 mg Inhalation Powder | | | |
|---|---|---|---|---|
| | 40° C./75% RH | | | |
| | Initial | 1 month | 3 months | 6 months |
| Nominal Dose (mg) | 5 | 5 | 5 | 5 |
| Delivered Dose (mg) | 4.3-4.8 | 4.0-4.6 | 3.9-4.4 | 3.9-4.4 |
| FPD ≦5 μm (mg) | 3.2-3.7 | 3.1-3.4 | 3.0-3.3 | 3.0-3.4 |
| FPF ≦5 μm (%) | 70-80 | 72-81 | 72-74 | 73-79 |
| FPD ≦3 μm (mg) | 2.7-3.2 | 2.6-2.9 | 2.5-2.7 | 2.5-2.8 |
| FPF ≦3 μm (%) | 59-68 | 61-70 | 60-62 | 60-66 |
| MMAD (μm) | 2.1 | 2.1 | 2.2 | 2.1-2.2 |

TABLE 20
Summary of Drug Delivery Performance, Size 3 HPMC
Capsules, Blister Packed. Test Device: Monohaler.

| | Sumatriptan Base 10 mg Inhalation Powder | | | |
|---|---|---|---|---|
| | 25° C./60% RH | | | |
| | Initial | 1 month | 2 months | 3 months |
| Nominal Dose (mg) | 10 | 10 | 10 | 10 |
| Delivered Dose (mg) | 9.1-10.0 | 8.2-9.8 | 9.3-10.2 | 8.9-10.2 |
| FPD ≦5 μm (mg) | 7.5-7.8 | 6.7-7.6 | 7.2-7.6 | 7.0-7.3 |
| FPF ≦5 μm (%) | 75-80 | 68-76 | 75-77 | 73-77 |
| FPD ≦3 μm (mg) | 6.0-6.4 | 5.4-6.3 | 5.6-6.0 | 5.5-5.9 |
| FPF ≦3 μm (%) | 60-66 | 55-63 | 58-61 | 57-62 |
| MMAD (μm) | 2.2-2.3 | 2.2-2.3 | 2.3-2.4 | 2.3 |

TABLE 21
Summary of Drug Delivery Performance, Size 3 HPMC
Capsules, Blister Packed. Test Device: Monohaler.

| | Sumatriptan Base 10 mg Inhalation Powder | | | |
|---|---|---|---|---|
| | 30° C./65% RH | | | |
| | Initial | 1 month | 2 months | 3 months |
| Nominal Dose (mg) | 10 | 10 | 10 | 10 |
| Delivered Dose (mg) | 9.1-10.0 | 9.3-10.2 | 8.6-9.5 | 8.0-9.9 |
| FPD ≦5 μm (mg) | 7.5-7.8 | 6.9-7.6 | 7.3-7.6 | 7.1-7.6 |
| FPF ≦5 μm (%) | 75-80 | 74-77 | 77-80 | 77-79 |
| FPD ≦3 μm (mg) | 6.0-6.4 | 5.6-6.0 | 5.9-6.0 | 5.6-6.1 |
| FPF ≦3 μm (%) | 60-66 | 58-61 | 61-64 | 62-63 |
| MMAD (μm) | 2.2-2.3 | 2.3 | 2.2-2.3 | 2.2-2.3 |

TABLE 22
Summary of Drug Delivery Performance, Size 3 HPMC
Capsules, Blister Packed. Test Device: Monohaler.

| | Sumatriptan Base 10 mg Inhalation Powder | | | |
|---|---|---|---|---|
| | 40° C./75% RH | | | |
| | Initial | 1 month | 2 months | 3 months |
| Nominal Dose (mg) | 10 | 10 | 10 | 10 |
| Delivered Dose (mg) | 9.1-10.0 | 8.9-9.4 | 8.5-9.5 | 8.6-9.6 |
| FPD ≦5 μm (mg) | 7.5-7.8 | 7.1-7.4 | 6.8-7.4 | 7.2-7.3 |
| FPF ≦5 μm (%) | 75-80 | 76-78 | 70-75 | 76-80 |
| FPD ≦3 μm (mg) | 6.0-6.4 | 5.8-6.0 | 5.4-5.9 | 5.9 |
| FPF ≦3 μm (%) | 60-66 | 61-63 | 55-60 | 62-64 |
| MMAD (μm) | 2.2-2.3 | 2.2-2.3 | 2.2-2.3 | 2.3 |

TABLE 23
Summary of Drug Delivery Performance, Size 3 HPMC Capsules,
Blister Packed at 200° C. Test Device: Monohaler.

| | Sumatriptan Base 15 mg Inhalation Powder | | | | | |
|---|---|---|---|---|---|---|
| | 25° C./60% RH | | | | | |
| | Initial | 1 month | 3 months | 6 months | 9 months | 12 months |
| Nominal Dose (mg) | 15 | 15 | 15 | 15 | 15 | 15 |
| Delivered Dose (mg) | 13.6-15.4 | 12.5-14.7 | 10.7-15.0 | 12.6-14.9 | 13.4-15.3 | 7.9-15.3 |
| FPD ≦5 μm (mg) | 5.2-6.3 | 4.9-5.8 | 5.1-5.3 | 4.8-5.7 | 4.7-5.9 | 4.9-5.5 |
| FPF ≦5 μm (%) | 33-43 | 32-38 | 34-36 | 31-38 | 32-38 | 34-37 |
| FPD ≦3 μm (mg) | N/A | 2.9-3.5 | 3.4-3.7 | 3.4-3.7 | 2.9-3.6 | 3.2-3.4 |
| FPF ≦3 μm (%) | N/A | 19-23 | 23-26 | 22-24 | 20-23 | 22-23 |
| MMAD (μm) | 2.9-3.0 | 2.9-3.0 | 2.9-3.0 | 2.8-2.9 | 2.9-3.1 | 2.9-3.0 |

TABLE 24
Summary of Drug Delivery Performance, Size 3 HPMC Capsules,
Blister Packed at 200° C. Test Device: Monohaler.

| | Sumatriptan Base 15 mg Inhalation Powder | | | |
|---|---|---|---|---|
| | 40° C./75% RH | | | |
| | Initial | 1 month | 3 months | 6 months |
| Nominal Dose (mg) | 15 | 15 | 15 | 15 |
| Delivered Dose (mg) | 13.6-15.4 | 13.7-15.3 | 13.6-14.8 | 12.0-15.2 |
| FPD ≦5 μm (mg) | 5.2-6.3 | 5.0-5.5 | 5.0-5.2 | 5.1-5.7 |
| FPF ≦5 μm (%) | 33-43 | 33-38 | 33-38 | 38-39 |
| FPD ≦3 μm (mg) | N/A | 3.0-3.6 | 3.4-3.7 | 3.2-3.8 |
| FPF ≦3 μm (%) | N/A | 20-25 | 23-26 | 24-25 |
| MMAD (μm) | 2.9-3.0 | 3.0-3.1 | 2.9-3.0 | 2.9-3.0 |

TABLE 25
Summary of Drug Delivery Performance, Size 3 HPMC Capsules, Lower
Blister Sealing Temperature (160° C.). Test Device: Monohaler.

| | Sumatriptan Base 15 mg Inhalation Powder | | | | | |
|---|---|---|---|---|---|---|
| | 25° C./60% RH | | | | | |
| | Initial | 1 month | 3 months | 6 months | 9 months | 12 months |
| Nominal Dose (mg) | 15 | 15 | 15 | 15 | 15 | 15 |
| Delivered Dose (mg) | N/A | N/A | N/A | N/A | N/A | N/A |
| FPD ≦5 μm (mg) | 11.6-12.6 | 11.1-11.2 | 11.9 | 11.6-11.8 | 11.4-11.5 | 11.5-12.0 |
| FPF ≦5 μm (%) | 77-83 | 76-77 | 78-80 | 78-79 | 79-80 | 79-80 |
| FPD ≦3 μm (mg) | 8.0-9.1 | N/A | 8.4 | 8.1-8.2 | 8.1-8.3 | 8.1-8.8 |
| FPF ≦3 μm (%) | 54-60 | N/A | 55-56 | 54-55 | 57 | 56-59 |
| MMAD (μm) | 2.5-2.7 | 2.5-2.7 | 2.6-2.7 | 2.6-2.7 | 2.6 | 2.5-2.6 |

TABLE 26
Summary of Drug Delivery Performance, Size 3 HPMC Capsules, Monohaler.
Lower Blister Sealing Temperature (160° C.). Test Device: Monohaler.

| | Sumatriptan Base 15 mg Inhalation Powder | | | | | |
|---|---|---|---|---|---|---|
| | 30° C./65% RH | | | | | |
| | Initial | 1 month | 3 months | 6 months | 9 months | 12 months |
| Nominal Dose (mg) | 15 | 15 | 15 | 15 | 15 | 15 |
| Delivered Dose (mg) | N/A | N/A | N/A | N/A | N/A | N/A |
| FPD ≦5 μm (mg) | 11.6-12.6 | 11.6-12.1 | 11.8-11.9 | 10.9-11.7 | 11.4-11.6 | 10.9-11.1 |
| FPF ≦5 μm (%) | 77-83 | 78-81 | 80-82 | 78-81 | 80 | 77-79 |
| FPD ≦3 μm (mg) | 8.0-9.1 | N/A | 8.4-8.6 | 7.9-8.2 | 7.6-8.2 | 7.4-8.0 |
| FPF ≦3 μm (%) | 54-60 | N/A | 58-59 | 56 | 54-56 | 53-56 |
| MMAD (μm) | 2.5-2.7 | 2.6 | 2.6 | 2.6 | 2.6-2.7 | 2.6-2.7 |

TABLE 27
Summary of Drug Delivery Performance, Size 3 HPMC Capsules, Monohaler.
Lower Blister Sealing Temperature (160° C.). Test Device: Monohaler.

| | Sumatriptan Base 15 mg Inhalation Powder | | | | | |
|---|---|---|---|---|---|---|
| | 40° C./75% RH | | | | | |
| | Initial | 1 month | 3 months | 6 months | 9 months | 12 months |
| Nominal Dose (mg) | 15 | 15 | 15 | 15 | 15 | 15 |
| Delivered Dose (mg) | N/A | N/A | N/A | N/A | N/A | N/A |
| FPD ≦5 μm (mg) | 11.6-12.6 | 10.2-10.6 | 10.7 | 10.3 | 8.0-8.9 | 6.8-8.9 |
| FPF ≦5 μm (%) | 77-83 | 76-79 | 77-79 | 77 | 64-71 | 54-68 |
| FPD ≦3 μm (mg) | 8.0-9.1 | N/A | 7.4-7.5 | 7.1-7.3 | 5.1-5.8 | 4.1-5.6 |
| FPF ≦3 μm (%) | 54-60 | N/A | 54-55 | 53-55 | 40-47 | 33-43 |
| MMAD (μm) | 2.5-2.7 | 2.6-2.8 | 2.6 | 2.7 | 2.8-2.9 | 3.0 |

TABLE 28
Summary of Drug Delivery Performance, Size 3 HPMC
Capsules, Blister Packed. Test Device: Monohaler.

| | Sumatriptan Base 15 mg Inhalation Powder | | | | | | |
|---|---|---|---|---|---|---|---|
| | 25° C./60% RH | | | | | | |
| | Initial | 1 month | 2 months | 3 months | 6 months | 9 months | 12 months |
| Nominal Dose (mg) | 15 | 15 | 15 | 15 | 15 | 15 | 15 |
| Delivered Dose (mg) | 13.3-15.0 | 14.0-15.7 | 13.3-14.9 | 13.0-15.0 | 12.2-14.8 | 12.1-15.5 | 7.3-15.1 |
| FPD ≦5 μm (mg) | 8.9-10.7 | 10.0-11.2 | 8.3-10.7 | 10.7-11.1 | 9.4-10.9 | 10.5-10.7 | 9.4-10.7 |
| FPF ≦5 μm (%) | 59-68 | 64-73 | 61-70 | 70-72 | 64-72 | 68-70 | 64-70 |
| FPD ≦3 μm (mg) | 7.2-8.2 | 7.6-8.8 | 7.2-8.4 | 8.2-8.4 | 7.2-9.0 | 8.2-8.6 | 7.5-8.6 |
| FPF ≦3 μm (%) | 47-53 | 49-57 | 48-56 | 54-55 | 49-60 | 53-57 | 51-56 |
| MMAD (μm) | 2.3-2.4 | 2.3-2.4 | 2.3-2.4 | 2.4 | 2.2-2.4 | 2.3-2.4 | 2.3-2.4 |

TABLE 29
Summary of Drug Delivery Performance, Size 3 HPMC
Capsules, Blister Packed. Test Device: Monohaler.

| | Sumatriptan Base 15 mg Inhalation Powder | | | | | | |
|---|---|---|---|---|---|---|---|
| | 30° C./65% RH | | | | | | |
| | Initial | 1 month | 2 months | 3 months | 6 months | 9 months | 12 months |
| Nominal Dose (mg) | 15 | 15 | 15 | 15 | 15 | 15 | 15 |
| Delivered Dose (mg) | 13.3-15.0 | 13.4-15.5 | 13.7-15.1 | 14.4-15.8 | 13.4-14.6 | 12.3-15.1 | 13.3-14.6 |
| FPD ≦5 μm (mg) | 8.9-10.7 | 10.1-12.2 | 10.8-11.9 | 10.3-10.9 | 11.5-12.2 | 10.4-11.2 | 10.6-11.1 |
| FPF ≦5 μm (%) | 59-68 | 67-80 | 74-80 | 68-69 | 76-80 | 68-72 | 68-74 |
| FPD ≦3 μm (mg) | 7.2-8.2 | 7.4-9.4 | 8.7-9.4 | 7.6-8.5 | 8.9-9.9 | 8.3-8.8 | 8.5-9.2 |
| FPF ≦3 μm (%) | 47-53 | 49-62 | 60-64 | 50-54 | 59-65 | 54-57 | 54-61 |
| MMAD (μm) | 2.3-2.4 | 2.4-2.5 | 2.3 | 2.3-2.5 | 2.2-2.3 | 2.3 | 2.3-2.3 |

TABLE 30
Summary of Drug Delivery Performance, Size 3 HPMC
Capsules, Blister Packed. Test Device: Monohaler.

| | Sumatriptan Base 15 mg Inhalation Powder | | | | |
|---|---|---|---|---|---|
| | 40° C./75% RH | | | | |
| | Initial | 1 month | 2 months | 3 months | 6 months |
| Nominal Dose (mg) | 15 | 15 | 15 | 15 | 15 |
| Delivered Dose (mg) | 13.3-15.0 | 14.1-15.3 | 14.1-15.1 | 11.1-15.3 | 12.1-15.5 |
| FPD ≦5 μm (mg) | 8.9-10.7 | 10.0-11.0 | 10.2-11.0 | 10.3-11.2 | 10.1-11.0 |
| FPF ≦5 μm (%) | 59-68 | 68-75 | 68-72 | 68-74 | 68-72 |
| FPD ≦3 μm (mg) | 7.2-8.2 | 7.7-8.6 | 7.8-8.6 | 8.2-8.8 | 7.6-8.9 |
| FPF ≦3 μm (%) | 47-53 | 52-60 | 52-56 | 54-57 | 51-58 |
| MMAD (μm) | 2.3-2.4 | 2.3-2.4 | 2.4-2.5 | 2.3-2.5 | 2.3-2.4 |

### Example 4

A double-blind, randomised, placebo-controlled, dose-escalation and subsequent open-label comparator pilot study in healthy subjects was conducted using the formulation disclosed in Example 3. The primary objective was to assess the safety and tolerability of sumatriptan inhalation powder administered via the inhaled route using a MonoHaler®. The secondary objectives were to define the dose of sumatriptan inhalation powder which results in a mean observed maximal venous plasma concentration (Cₘₐₓ) of approximately 72 ng/mL. This target plasma concentration is the known Cₘₐₓ of the 6 mg subcutaneous sumatriptan, which is regarded as the gold standard of migraine treatments. Also to evaluate the safety, tolerability and pharmacokinetics of sumatriptan inhalation powder compared to that of subcutaneous sumatriptan in healthy subjects.

In Vivo Methodology (Part I)

The delivered doses administered in Part I were: Period 1: 2 mg, Period 2: 5 mg, Period 3: 10 mg, Period 4: 15 mg of sumatriptan inhalation powder or placebo (ratio 9:3) as inhalation delivered via Monohaler®.

As previously discussed, following inhalation there is potential for high, transient concentrations in the pulmonary vein and coronary vasculature. The previous method of studying the effect of triptans on the coronary vasculature is that as published by Hillis. (Maclntyre, P D, Bhargava B, Hogg K J, Gemmill J D and Hillis W S, Circulation 1993 87 401-405). This involves patients undergoing diagnostic coronary angiography and invasive haemodynamic monitoring. The difficulty in using this procedure for an inhalation product is that the patients must remain semi-supine which will affect the deposition pattern of the drug. In addition, as this type of study involves patients with suspected cardiac disease, (ethically the procedure is unlikely to allowed on healthy subjects), there is a degree of risk. Therefore for this clinical study arterial blood sampling was used in a novel application, as the closest surrogate measure of the concentrations experienced by the coronary vasculature.

In Vivo Data Part I

Pharmacokinetic Results:

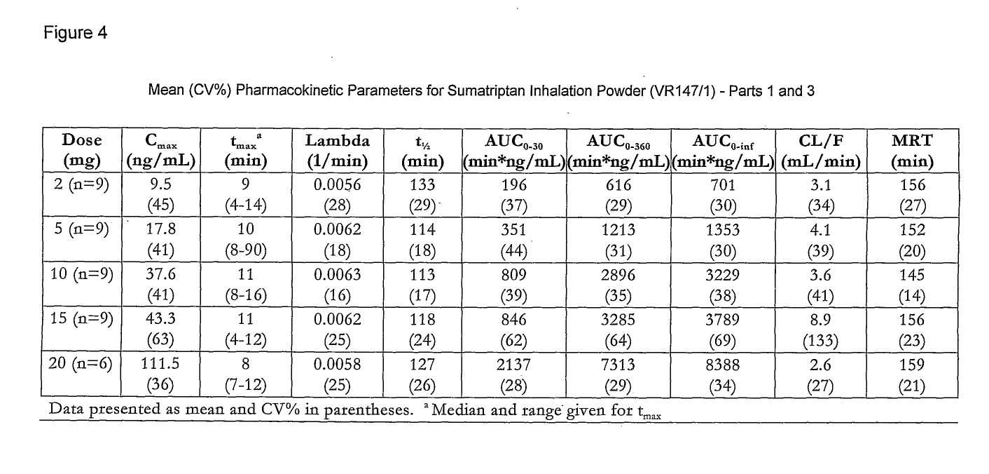

The table of data in FIG. 4 shows a summary of the venous pharmacokinetic data—Escalating Doses from 2 to 15 mg inhaled sumatriptan [mean (CV %) except Tₘₐₓ reported as a median (range)]. The target Cₘₐₓ of 72 ng/mL was not achieved within this dose range.

Table 32 shows a comparison between arterial and venous PK data. The arterial Cₘₐₓ are 70% higher than the venous level in the same subject. This may indicate a lower dose than indicated by the venous data will be efficacious, as the arterial levels are more relevant to the site of action, the brain, than the venous levels. The arterial Tₘₐₓ is earlier than seen with the venous sampling. This suggests that the onset of effect following inhalation may be earlier than expected from the venous data.

TABLE 32

| | | | | | | AUC₀₋₁₀ |
|---|---|---|---|---|---|---|
| | | Cₘₐₓ (ng/mL) | | tₘₐₓ (min) | | (min*ng/mL) |
| Dose | Subject | Arterial | Venous | Arterial | Venous | Arterial |
| 2 mg | 102 | 15.8 | 7.86 | 6 | 12 | 125 |
| | 111 | 12.9 | 6.33 | 3 | 4 | 97 |
| 5 mg | 101 | 37.6 | 25.4 | 4 | 8 | 321 |
| | 107 | 33.4 | 18.6 | 4 | 12 | 262 |
| 10 mg | 108 | 97.3 | 59.6 | 5 | 9 | 805 |
| | 110 | 27.2 | 21.6 | 4 | 11 | 221 |

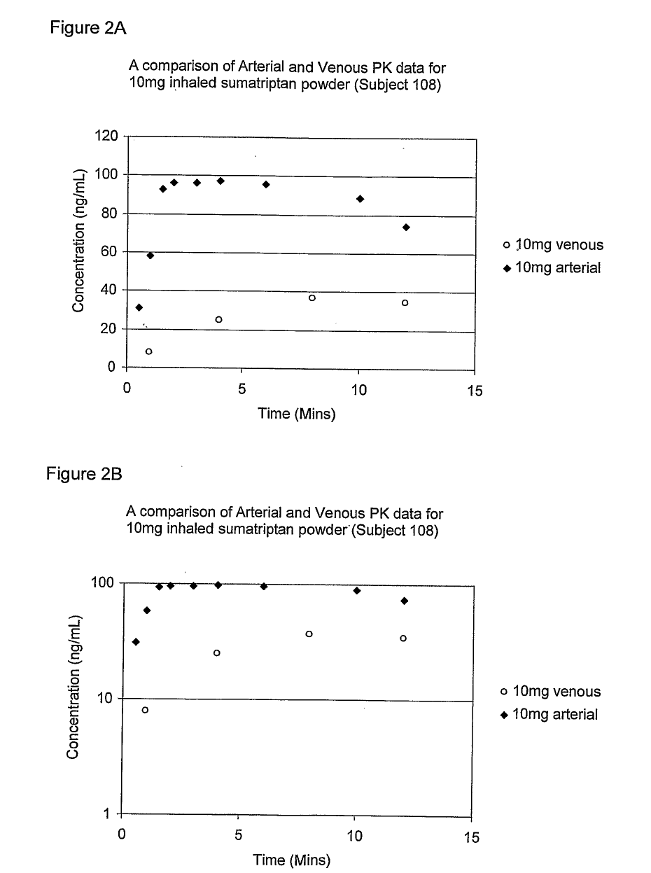

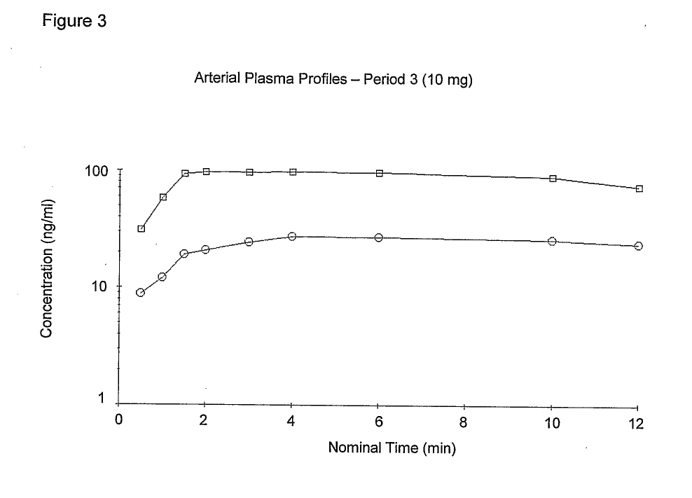

FIG. 2A shows the arterial and venous plasma profiles following administration of a 10 mg dose to subject 108 of sumatriptan administered by pulmonary inhalation. FIG. 2B shows a logarithmic plot of arterial plasma profiles of FIG. 2A. FIG. 3 compares the arterial plasma profiles following administration of a 10 mg dose in subject 110(circles) and subject 108 (squares).

FIGS. 2 and 3 demonstrate a clear depot effect. The concentration increases over the first few minutes and then remains steady for the next ten minutes, rather than just dropping immediately as the drug is diluted by the circulation. The inhaled sumatriptan resides within the airways exhibiting a depot effect which translates into blood levels approximately 1.5 times higher in the arterial system than that as observed in the venous system. The arterial data demonstrates that lung acts as a reservoir leaching drug into the pulmonary vein over a period of approximately 10 minutes following the administration of the drug.

In Vivo Methodology (Part II)

A 2 way crossover randomised,single dose comparison of inhaled sumatriptan (15 mg) and subcutaneous sumatriptan (6 mg) was conducted. Part II was planned as an open-label comparison of the target dose of sumatriptan inhalation powder established in Part I and subcutaneous sumatriptan. As the target Cₘₐₓ was not reached in Part 1, the top dose, (15 mg) was compared with subcutaneous sumatriptan (6mg) in a randomised two-way crossover design.

In Vivo Data Part II

Pharmacokinetic Results:

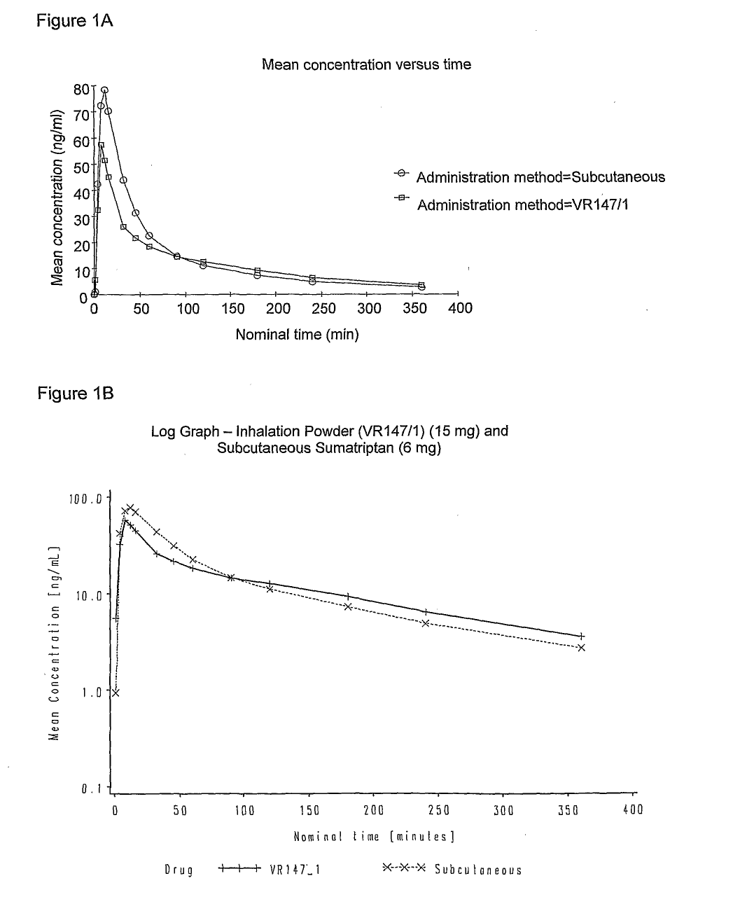

Pharmacokinetic data for inhaled sumatriptan (15 mg) and subcutaneous sumatriptan (6 mg) are summarised in Table 31. Graphs of the results are shown in FIGS. 1A and B, which show the mean plasma concentration profiles (Linear and Log Scale) following subcutaneous sumatriptan and inhaled sumatriptan. The pharmacokinetic curve for administration by inhalation is similar to that for subcutaneous administration (see FIG. 1), which is surprising as one might expect an earlier concentration peak following inhalation. This may be explained by the depot effect described above. The similarity in PK profile shape suggests the inhaled sumatriptan powder may show similar high levels of consistency and efficacy to the subcutaneous route. The subcutaneous route is known to be associated with the greatest consistency and efficacy, in comparison with the alternative routes of administration (‘The Triptans Novel Drugs for Migraine’ ed Humphrey P., Ferrari M, and Oleson J. 2001). This is due to the variable absorption from other sites, which leads to multiple peaks in the PK curves. The inter-subject variability of the majority of PK parameters was slightly greater for the inhaled sumatriptan than subcutaneous, but not too dissimilar. The variability is much less than would be seen for the other routes of administration. For example, the coefficient of variation for Tₘₐₓ for the intranasal route is 62.8% (Duquesnoy et al European Journal of Pharmaceutical Sciences 6 (1998) 99-104), compared with 30.3% for inhaled sumatriptan from this study. The variability in the nasal route is due to multiple peaks in the PK curve, probably due to some of the dose being swallowed. Similarly with the oral route, changes in the rate of gastric emptying cause high variability.

TABLE 31
Comparison of PK Parameters Between Sumatriptan Inhalation Powder
(VR147/1) (15 mg) and Subcutaneous Sumatriptan (6 mg)

| | | |
|---|---|---|
| | Sumatriptan Inhalation | Subcutaneous |
| | Powder | Sumatriptan |
| Parameter | (15 mg) (n = 12) | (6 mg) (n = 12) |

| | | | | |
|---|---|---|---|---|
| Cₘₐₓ (ng/mL) | 59.8 | (35.2) | 80.4 | (33.4) |
| tₘₐₓ (min) | 8 | (7-17) | 12 | (8-16) |
| t_{1/2} (min) | 119 | (21.5) ¹ | 111 | (12.1) ¹ |
| AUC₀₋₃₀ | 1155 | (33.3) | 1688 | (29.8) |
| (min*ng/mL) | | | | |
| AUC₀₋₃₆₀ | 4385 | (31.5) | 4911 | (16.6) |
| (min*ng/mL) | | | | |
| AUC_{0-inf} | 5087 | (35.3) ¹ | 5290 | (18.5) ¹ |
| (min*ng/mL) | | | | |
| V_{z}/F (L) | 548 | (30.8) ¹ | 185 | (17.2) ¹ |
| CL/F (L/min) | 3.28 | (33.1) ¹ | 1.18 | (20.6) ¹ |

Data presented as mean and CV % in parentheses. Median and range given for t_{max.}
¹ Data from 11 subjects (excludes Subject 109). For subject 109 it was not possible to estimate the half life due to an increase in plasma concentration at the final timepoint of both periods.

The time taken to reach peak concentration (Tₘₐₓ) was shorter for the inhaled product (median Tₘₐₓ 8, range 7 to 17 minutes for inhaled versus 12, range 8 to 16 minutes for subcutaneous: Table 31). The timing of the onset of action is thought to be related to Tₘₐₓ. For the subcutaneous injection the onset of action is 10 minutes. If the arterial Tₘₐₓ for the inhaled product is considered in comparison with the subcutaneous venous Tₘₐₓ, then it is possible the inhaled sumatriptan powder would show an earlier onset of effect than the subcutaneous product. If this were to be proven, this would make the inhaled route the fastest acting treatment for migraine available. Sumatriptan half-life was comparable for the 2 treatments; for the inhaled product mean T_{1/2} was 119 minutes whilst for subcutaneous treatment it was 111 minutes. This suggests that the duration of effect will be similar for the inhaled product to the subcutaneous injection, as this is related to half life. (Geraud G, Keywood C and Senard J M headache 2003 43 376-388).

Indeed, the AUC figures indicate that the inhaled dose of sumatriptan will have a similar duration of effect to that seen following a subcutaneous administration. This is surprising as administration of active agents by inhalation is often characterised by a rapid onset of the therapeutic effect followed by a rapid offset.

In Vivo Methodology (Part III)

As the target Cₘₐₓ was not reached in Part I, it was estimated from the data a dose of 20 mg would be required to reach this plasma concentration. Therefore a further delivered dose of 20 mg of sumatriptan inhalation powder was given to eight of the volunteers. They received two sequential inhaled doses of 10 mg sumatriptan (giving a total dose of 20 mg), or placebo (ratio 6:2), as inhalation delivered via the Monohaler®.

In Vivo Data

Pharmacokinetic Results:

At the 20 mg delivered dose the mean Cₘₐₓ is 112 ng/mL, which exceeds the target plasma concentration (FIG. 4) and was higher than expected from extrapolation of the Part I and II data. Therefore based on the venous data it may be suggested a dose between 15 and 20 mg will provide similar efficacy to that of the 6 mg subcutaneous injection.

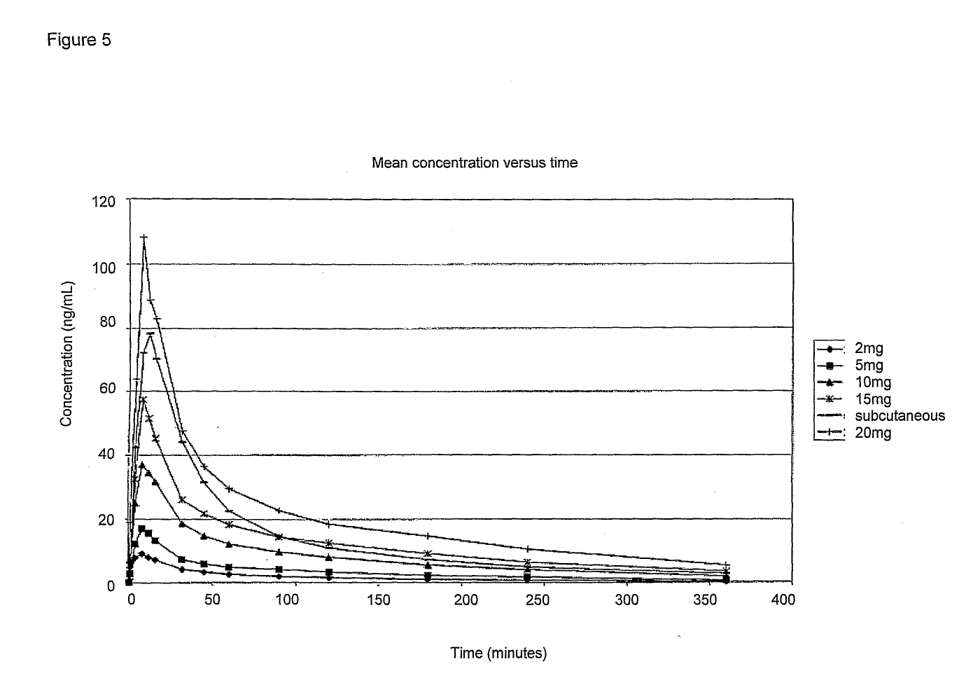

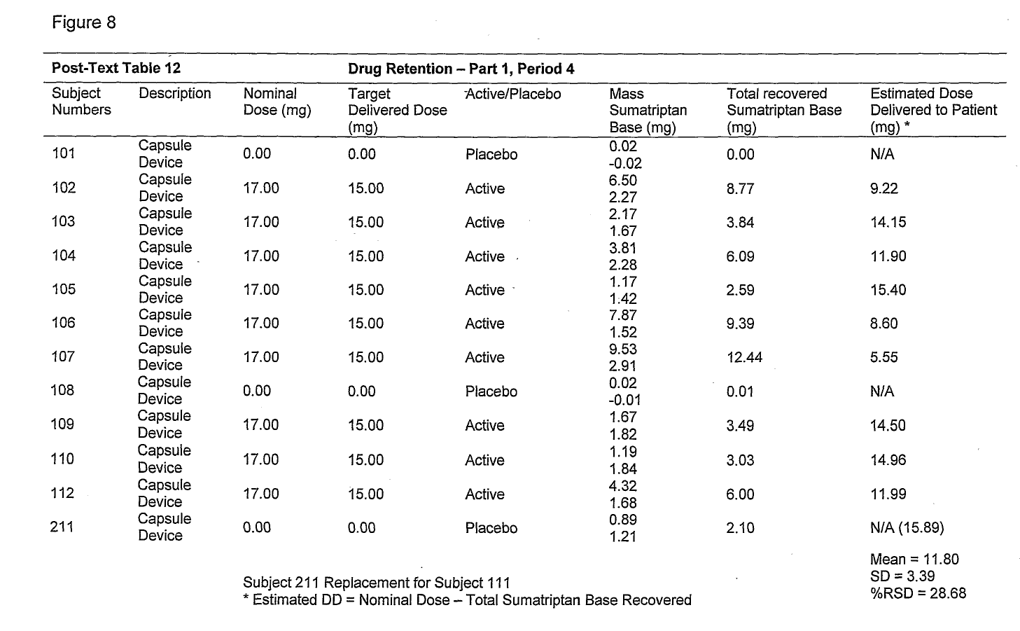

A graph of the results from Part I, II and III is shown in FIG. 5, which shows the mean plasma concentration profiles following subcutaneous sumatriptan and inhaled sumatriptan at the trialled doses. The plasma levels from the 15 mg dose in Part I were lower than expected, which was subsequently found to be due to three subjects not inhaling correctly. Evidence for this was found from analysis of the drug retained in the used capsules (FIG. 8). When the data is reanalysed excluding these subjects, the mean Cₘₐₓ is very close to that obtained for the same dose in Part II.

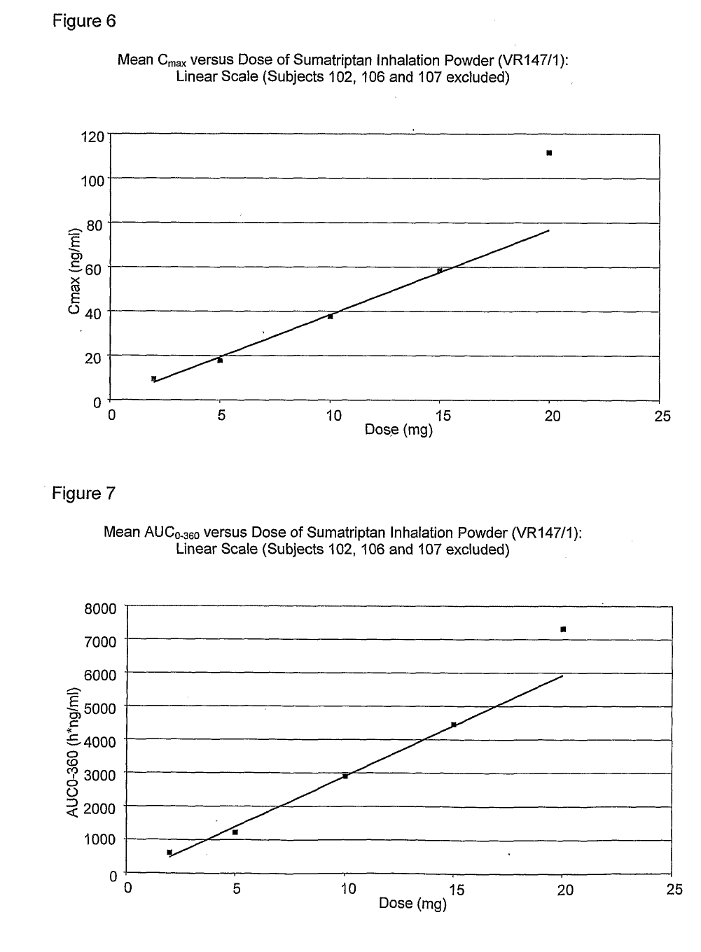

FIGS. 6 and 7 plot Cₘₐₓ and AUC against dose for Parts I and III. These illustrate the lines of best fit including the data from the 15 mg cohort from Part I (excluding 3 subjects). The 20 mg dose is above the line and supraproportional, therefore the dose proportionality was assessed excluding this dose.

TABLE 34
Dose Proportionality (excluding Subject
102, 106 and 107, 15 mg data)

| | Slope | | | | |
|---|---|---|---|---|---|
| Parameter | Estimate | SE | Lower 95% CI | Upper 95% CI | P-value |
| Cₘₐₓ | 0.92 | 0.111 | 0.695 | 1.148 | <.0001 |
| AUC₀₋₃₆₀ | 0.99 | 0.081 | 0.822 | 1.152 | <.0001 |

From the study (Table 34), it could be concluded that, for the inhaled product, an approximately linear relationship has been confirmed between dose and Cₘₐₓ and between dose and AUC₍₀₋₃₆₀₎ over the dose range 2 to 15 mg. The high Cₘₐₓ and exposure following the 20 mg dose is surprising because this dose was given as two capsules, which would be expected to be result in greater losses upon delivery, for example in retention in the capsule. It is possible that it is easier to inhale the powder dose as two separate capsules and that this therefore is an advantage to splitting the dose.

The inter-subject variability in Cₘₐₓ was less for the 20 mg (2 times 10 mg) dose than for any of the other inhaled doses (FIG. 4, the coefficient of variation for 20 mg was 36% compared with 41-63% for the other doses). This is surprising given that the sumatriptan is being administered as two inhalations.

Safety Results (Parts I, II and III)

It could be concluded from the studies that there were no safety or tolerability concerns with up to and including 20 mg of the inhaled sumatriptan formulation (or placebo) in the study population.

All subjects experienced treatment emergent adverse events (TEAEs) that were typical for triptans. The TEAE incidence was comparable following the highest dose of sumatriptan inhalation powder (20 mg) administered (13 events in 6 subjects) with standard subcutaneous sumatriptan (21 events in 12 subjects). Most adverse events were mild, occurred in the first hour after dosing and were resolved within an hour. Surprisingly, there were no significant abnormalities or trends in the 12-lead ECGs or the 12 lead holter tapes. There were no consistent or clinically significant effects on FEV1 or SpO2, with the exception of one subject. This subject was found to be mildly asthmatic and was withdrawn from the study.

The absence of adverse cardiovascular system side effects is surprising in light of the high levels of sumatriptan measured in the pulmonary vein following administration of the drug by pulmonary inhalation. None of the patients that took sumatriptan by inhalation suffered from nausea or vomiting.

### Example 4

### pMDIs

Formulation Methodology

Preparation of pMDIs: The powders comprising pure micronised sumatriptan succinate were measured into pMDI cans. Metering valves were clamped onto the cans, and these were back filled with HFA 134a propellant Each can was shaken vigorously to generate a dispersion.

In vitro measurement of pMDIs: An Andersen cascade impactor was used to characterise the aerosol plumes generated from each of the pMDIs. Air-flow of 28.3 litres per minute was drawn through the impactor, and 10 repeated shots were fired. Each pMDI was shaken and weighed in between each actuation. The drug deposited on each stage of the impactor, as well as drug on the device, throat and rubber mouthpiece adaptor was collected into a solvent, and quantified by HPLC.

The low solubility of sumatriptan succinate within ethanol-based HFA 134a pMDI formulations makes solution pMDI technology unavailable for sumatriptan at high drug loading. Previously a low dose (<25 μg/50 HFA 134a/HFA 227 solution formulation has been produced but only at high ethanol contents (50% w/w). A sumatriptan analogue may be used to formulate highly efficient solution formulations at the desirable dose range of 100 to 500 μg/50 μl.

1. A pharmaceutical composition comprising a triptan, for administration by pulmonary inhalation.

2. A composition as claimed in claim 1, wherein the triptan is sumatriptan.

3. A composition as claimed in claim 1, for treatment or prophylaxis of conditions of the central nervous system, including migraine.

4. A composition as claimed in claim 1, comprising a dose of sumatriptan succinate at least 1 mg and up to 15 mg, up to 20 mg or up to 25 mg.

5. A composition as claimed in claim 1, wherein the composition provides a fine particle dose (FP D) of about 2 to about 16 mg upon administration.

6. A composition as claimed in claim 1, wherein (a) doses may be administered sequentially, with the effect of each dosing being assessed by the patient before the next dose is administered to allow self-titration and optimisation of the dose, and/or (b) doses of the sumatriptan succinate composition are to be administered to the patient as needed.

7. A composition as claimed in claim 1, wherein the composition provides a daily dose, which is the dose administered over a period of 24 hours, of between about 0.5 and about 25 mg.

8. A composition as claimed in claim 1, wherein the composition allows doses to be administered at regular and frequent intervals providing maintenance therapy.

9. A composition as claimed in claim 1, wherein the composition provides a mean Cₘₐₓ within less than about 10 minutes of administration by pulmonary inhalation.

10. A composition as claimed in claim 1, wherein the composition provides a dose dependent Cₘₐₓ upon administration by pulmonary inhalation.

11. A composition as claimed in claim 1, wherein the composition provides a therapeutic effect in about 10 minutes or less following administration by pulmonary inhalation.

12. A composition as claimed in claim 1, wherein wherein the composition comprises at least about 70% (by weight) sumatriptan succinate.

13. A composition as claimed in claim 1, further comprising an additive material.

14. A composition as claimed in claim 1, further comprising particles of an inert excipient material.

15. A blister or capsule containing a composition as claimed in claim 1.

16. An inhaler device comprising a composition as claimed in claim 1.

17. An inhaler device as claimed in claim 15, wherein the device is a dry powder inhaler, a pressurized metered dose inhaler or a nebuliser.

18. The process of using a composition as claimed in claim 1 in the manufacture of a medicament for treating diseases of the central nervous system, such as migraine, tension type headache or cluster headache by pulmonary inhalation.

19. A process for comprising preparing a composition as claimed in claim 1.

20. A process as claimed in claim 19, wherein the triptan is spray dried.

21. A method of treating migraine in a human via inhalation, comprising: inhaling a dose of a powder composition as claimed in claim 1.

22. A pharmaceutical composition comprising a triptan, for administration by pulmonary inhalation, wherein said composition is to be administered in at least two sequential doses.

23. A pharmaceutical composition as claimed in claim 22, wherein the sequential doses are to be administered within a period of no more than 5 minutes.

24. A pharmaceutical composition as claimed in claim 22, wherein the sequential doses are of substantially the same size.

25. A pharmaceutical composition as claimed in claim 22, wherein only two sequential doses are to be administered.

26. A pharmaceutical composition as claimed in claim 22, wherein said sequential doses are sufficient to provide a maximum serum concentration (Cₘₐₓ) of triptan that is in excess of double that provided by the administration of the first or a single such dose of the triptan when administered alone to the same subject.

27. A pharmaceutical composition as claimed in claim 22, wherein each administered dose is of between 5 and 15 mg, 8 and 12 mg, 9 and 11 mg, 9.5 and 10.5 mg or about 10 mg of triptan.

28. A pharmaceutical composition as claimed in claim 22, wherein the triptan is sumatriptan.

29. A pharmaceutical composition as claimed in claim 27, wherein the doses are metered doses or nominal doses, or, alternatively, delivered doses or emitted doses.

30. A pharmaceutical composition as claimed in claim 22, for the treatment or prophylaxis of a condition of the central nervous system.

31. A pharmaceutical composition as claimed in claim 22, for the treatment or prophylaxis of migraine.

32. A method of treating a subject in need of therapy with a triptan, comprising administering to said subject a pharmaceutical composition comprising an effective amount of a triptan by pulmonary inhalation, wherein said composition is administered to said subject in at least two sequential doses.

33. A method as claimed in claim 32, wherein the sequential doses are administered within a period of no more than 5 minutes.

34. A method as claimed in claim 32, wherein the sequential doses are of substantially the same size.

35. A method as claimed in claim 32, wherein only two sequential doses are administered.

36. A method as claimed in claim 32, wherein said sequential doses are sufficient to provide a maximum serum concentration (Cₘₐₓ) of triptan that is in excess of double that provided by the administration of the first or a single dose of the triptan when administered alone to the same subject.

37. A method as claimed in claim 32, wherein each administered dose is of between 5 and 15 mg, 8 and 12 mg, 9 and 11 mg, 9.5 and 10.5 mg or about 10 mg of triptan.

38. A method as claimed in claim 32, wherein the triptan is sumatriptan.

39. A method as claimed in claim 37, wherein the doses are metered doses or nominal doses, or, alternatively, delivered doses or emitted doses.

40. A method as claimed in claim 32, for the treatment or prophylaxis of a condition of the central nervous system.

41. A method as claimed in claim 32, for the treatment or prophylaxis of migraine.

42. A method, use, product, process or composition as claimed claim 1, wherein, when it is not sumatriptan, the triptan is rizatriptan, naratriptan, zolmitriptan, eletriptan, almotriptan or frovatriptan.
